(19) United States
(12) Patent Application Publication
(10) Pub. No.: US 2022/0031713 A1
(43) Pub. Date: Feb. 3, 2022

(54) METHODS OF TREATING MYELOPROLIFERATIVE DISORDERS

(71) Applicant: Impact Biomedicines, Inc., Summit, NJ (US)

(72) Inventors: Torsten Guenter Gerike, Summit, NJ (US); Aleksandra Rizo, Mount Kisco, NY (US)

(21) Appl. No.: 17/279,763

(22) PCT Filed: Sep. 24, 2019
(86) PCT No.: PCT/US2019/052607

§ 371 Date: Mar. 25, 2021

Related U.S. Application Data
(60) Provisional application No. 62/736,349, filed on Sep. 25, 2018.

(51) Int. Cl. A61K 31/635

(52) U.S. Cl. CPC A61K 31/635

(57) ABSTRACT

The present disclosure provides methods of treating a myeloproliferative disorder. In some aspects, the present disclosure provides methods of treating, stabilizing or lessening the severity or progression of one or more myeloproliferative disorders comprising administering to a patient previously treated with ruxolitinib a pharmaceutically acceptable composition comprising a compound of formula I, also known as fedratinib, or a pharmaceutically acceptable salt or hydrate thereof.

### CROSS-REFERENCE TO RELATED APPLICATIONS

The present application claims priority to U.S. Provisional Patent Application No. 62/736,349, filed on Sep. 25, 2018, the entirety of which is hereby incorporated by reference.

### FIELD OF THE INVENTION

The present invention provides methods of treating, stabilizing or lessening the severity or progression of a myeloproliferative disorder.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

The present disclosure provides methods of treating, stabilizing or lessening the severity or progression of one or more myeloproliferative disorders. In certain embodiments, the present disclosure provides methods of treating a patient previously treated with ruxolitinib (JAKAFI®; (3R)-3-cyclopentyl-3-[4-(7H-pyrrolo[2,3-d]pyrimidin-4-yl)pyrazol-1-yl]propanenitrile).

In some aspects, the present disclosure provides methods of treating, stabilizing or lessening the severity or progression of one or more myeloproliferative disorders comprising administering to a patient previously treated with ruxolitinib a pharmaceutically acceptable composition comprising a compound of formula I:

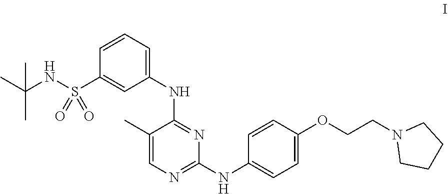 [SMILES: Cc1cnc(Nc2ccc(OCCN3CCCC3)cc2)nc1Nc1cccc(S(=O)(=O)NC(C)(C)C)c1]

or a pharmaceutically acceptable salt or hydrate thereof. The compound of formula I is also referred to herein as “Compound I”. In some embodiments, Compound I is in the form of a dihydrochloride salt. Compound I, or a pharmaceutically acceptable salt thereof, may also exist in a hydrate form. In some embodiments, Compound I is in the form of a dihydrochloride monohydrate. Accordingly, in some embodiments, provided methods comprise administering to a patient in need thereof Compound II:

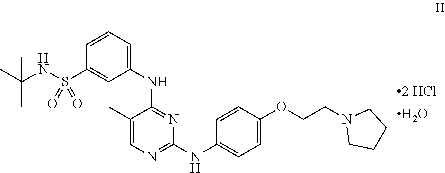

In some embodiments, the present disclosure provides a method of treating a myeloproliferative disorder, comprising administering to a patient previously treated with ruxolitinib Compound I, or a pharmaceutically acceptable salt or hydrate thereof, (e.g., Compound II).

In some embodiments, the patient has been previously treated with ruxolitinib for at least 3 months. In some embodiments, the patient has been previously treated with ruxolitinib for at least 3 months with inadequate efficacy response defined as <10% spleen volume reduction by MM or <30% decrease from baseline in spleen size by palpation or regrowth to these parameters following an initial response. Patients who experience inadequate efficiency are said to be refractory. Patients who experience regrowth to those parameters are said to be relapsed.

In some embodiments, the patient has been previously treated with ruxolitinib for at least 28 days complicated by
i. development of a red blood cell transfusion requirement; or
ii. Grade ≥3 adverse event(s) of thrombocytopenia, anemia, hematoma, and/or hemorrhage while on treatment with ruxolitinib.

In some embodiments, the present disclosure provides a method of reducing spleen volume by at least 25% in a patient suffering from or diagnosed with a myeloproliferative disorder. In some embodiments, the patient's spleen volume is reduced by at least 35%. In some embodiments, spleen volume is measured by magnetic resonance imaging (MM) or computed tomography (CT).

In some embodiments, the present disclosure provides a method of improving overall survival in a patient suffering from or diagnosed with a myeloproliferative disorder. In some embodiments, the overall survival is improved relative to best available therapy.

In some embodiments, the present disclosure provides a method of treating a patient that is suffering from or diagnosed with a myeloproliferative disorder that is resistant or refractory to ruxolitinib. In some embodiments, the patient has exhibited or experienced one or more of the following during treatment with ruxolitinib: lack of response, disease progression, or loss of response/therapeutic effect. In some embodiments, disease progression is evidenced by an increase in spleen size during ruxolitinib treatment.

In some embodiments, the present disclosure provides a method of treating a myeloproliferative disorder in a patient who is intolerant to ruxolitinib. In some embodiments, intolerance to ruxolitinib is evidenced by a hematological toxicity (e.g., anemia, thrombocytopenia, etc.) or a non-hematological toxicity.

In some embodiments, the present disclosure provides a method of treating a myeloproliferative disorder in a patient previously treated with ruxolitinib, wherein the patient has relapsed.

In some embodiments, the present disclosure provides a method of improving symptom response rate in a patient suffering from or diagnosed with a myeloproliferative disorder. In some such embodiments, symptom response rate is evidenced by at least 50% reduction in total symptom score (TSS), as defined infra. In some embodiments, the symptom response rate is improved relative to best available therapy.

In some embodiments, the present disclosure provides a method of increasing the median survival in patients who have relapsed or are refractory to ruxolitinib. In some embodiments, the median survival is increased relative to best available therapy.

Activating mutations in the pseudokinase domain of JAK2 occur at a high frequency in Philadelphia chromosome-negative myeloproliferative disorders. Increasing JAK2 V617F allele burden has been shown to correlate with disease severity (bone marrow dysfunction, organomegaly and constitutional symptoms), which is consistent with exaggerated JAK2 signaling playing a central role in myeloproliferative disorders. Accordingly, in some embodiments, the present disclosure provides a method of decreasing allele burden in a patient having a somatic mutation or clonal marker associated with or indicative of a myeloproliferative disorder. In some embodiments, the somatic mutation is selected from a JAK2 mutation, a calreticulin (CALR) mutation or a myeloproliferative leukemia virus (MPL) mutation. In some embodiments, the JAK2 mutation is V617F. In some embodiments, the CALR mutation is a mutation in exon 9. In some embodiments, the MPL mutation is selected from W515K and W515L. In some embodiments, the allele burden is decreased relative to the patient's allele burden prior to treatment with Compound I, or a pharmaceutically acceptable salt or hydrate thereof.

In some embodiments, a myeloproliferative disorder is selected from intermediate risk MPN-associated myelofibrosis and high risk MPN-associated myelofibrosis.

In some embodiments, the intermediate risk MPN-associated myelofibrosis is selected from primary myelofibrosis, post-polycythemia vera (post-PV) myelofibrosis and post-essential thrombocythemia (post-ET) myelofibrosis.

In some embodiments, the high risk MPN-associated myelofibrosis is selected from primary myelofibrosis, post-polycythemia vera (post-PV) myelofibrosis and post-essential thrombocythemia (post-ET) myelofibrosis.

In some embodiments, provided methods induce a complete response (CR), as defined infra. In some embodiments, provided methods induce a partial response, as defined infra. In some embodiments, provided methods induce a clinical improvement, as defined infra. In some embodiments, provided methods induce a spleen response, as defined infra.

In some embodiments, the present disclosure provides a method of treating a myeloproliferative disorder in a patient previously treated with ruxolitinib, wherein the patient is administered about 400 mg of Compound I. In some embodiments, the dose of Compound I is decreased from about 400 mg to about 300 mg. In some embodiments, the dose of Compound I is decreased from about 300 mg to about 200 mg. In some embodiments, Compound I is administered once a day for one or more 28-day cycles. In some embodiments, Compound I is administered once a day for at least six 28-day cycles.

In some embodiments, the present disclosure provides a method of minimizing one or more adverse events relating to or resulting from treatment with Compound I. In some embodiments, the patient is at risk of developing Wernicke's encephalopathy. In some such embodiments, the patient is monitored for Wernicke's encephalopathy.

### Definitions

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. As an example, when the term “about” is used in combination with a certain number of days, it includes said specific number of days plus or minus 1 day, e.g., “about 6 days” includes any number of days between 5 and 7. It is to be understood that the value to which the modifier “about” refers is itself also specifically, and preferably, disclosed.

The terms “treat” or “treating,” as used herein, refers to partially or completely alleviating, inhibiting, delaying onset of, preventing, ameliorating and/or relieving a disorder or condition, or one or more symptoms of the disorder or condition. As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of inventive formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### DETAILED DESCRIPTION OF CERTAIN EMBODIMENTS

Myelofibrosis

Myeloproliferative neoplasm (MPN)-associated myelofibrosis (MF) is a serious and life-threatening disease that can present as a de novo or primary myelofibrosis (PMF) or evolve from previous polycythemia vera or essential thrombocythemia (Swerdlow S H, Campo E, Harris N L, Jafie E S, Pileri S A, Stein H, et al. World Health Organization classification of tumors of haematopoietic and lymphoid tissues. Lyon: IARC Press 2008). The disease is characterized by clonal myeloproliferation, ineffective erythropoiesis, bone marrow stromal changes, hepatosplenic extramedullary hematopoiesis, and aberrant cytokine expression (Tefferi A, Pardanani A. JAK inhibitors in myeloproliferative neoplasms: rationale, current data and perspective. Blood Rev. 2011 September; 25(5):229-37). Patients typically present with splenomegaly, constitutional symptoms, moderate to severe anemia, thrombocytopenia, and leukocytosis.

Primary myelofibrosis is a member of a group of Philadelphia chromosome (Ph1)-negative MPNs which also includes polycythemia vera (PV) and essential thrombocythemia (ET) (Tefferi A. The recent advances in classic BCR-ABL-negative myeloproliferative disorders. Clin. Adv. Hematol. Oncol. 2007a; 5:113-5). Almost all patients with PV and about one-half of patients with ET and PMF have a JAK2 mutation, typically JAK2V617F. Other mutations in patients with PMF include CALR and MPL. About 20% of patients with PMF have no detectable mutation in JAK2, CALR, or MPL and are termed triple negative (Levine R L, Wadleigh M, Cools J, Ebert B L, Wernig G, Huntly B J, et al. Activating mutation in the tyrosine kinase JAK2 in polycythemia vera, essential thrombocythemia, and myeloid metaplasia with myelofibrosis. Cancer Cell. 2005; 7:387-97; Werning G, Mercher T, Okabe R, Levine L, Lee B H, Gilliland G L. Expression of JAK2V617F causes a polycythemia vera-like disease with associated myelofibrosis in a murine bone marrow transplant model. Blood. 2006; 107:4274-81). Mutations in JAK2, CALR, and MPL result in activation of the JAK/STAT signaling pathway resulting in cell proliferation and inhibiting cell death. The result is clonal expansion (Ilhe J N, Gilliland D G. JAK2: normal function and role in hematopoietic disorders. Curr. Opin. Genet. Dev. 2007; 17:8-14). Thus, a JAK2 inhibitor that can down regulate the JAK/STAT pathway is expected to be helpful in reducing cell proliferation.

Polycythemia vera (PV) and essential thrombocythemia (ET) are characterized by increased levels of red blood cells (RBC) and platelets. However, about 10% of affected patients develop bone marrow fibrosis morphologically indistinguishable from PMF. These conditions are termed post-polycythemia vera myelofibrosis (post-PV-MF) and post-essential thrombocythemia myelofibrosis (post-ET-MF) (Campbell P J, Green A R. Management of polycythemia vera and essential thrombocythemia. Hematology Am. Soc. Hematol. Educ. Program. 2005; 201-8), and are clinically named MPN-associated myelofibrosis. Patients with MPN-associated myelofibrosis have similar survival prognoses to that of the PMF and about a 10% cumulative risk of transformation to acute myeloid leukemia (AML).

There are several prognostic scoring systems predicting survival of patients with PMF. The International Prognostic Scoring System (IPSS) is used to predict survival at diagnosis and the Dynamic International Prognostic Scoring System (DIPSS) at any time in the disease course (Cervantes F, Dupriez B, Pereira A, et al. New prognostic scoring system for primary myelofibrosis based on a study of the International Working Group for Myelofibrosis Research and Treatment. Blood. 2009; Mar. 26; 113(13):2895-901; Passamonti F, Cervantes F, Vannucchi A M, Morra E, Rumi E, Pereira A, et al. A dynamic prognostic model to predict survival in primary myelofibrosis: a study by the IWG-MRT (International Working Group for Myeloproliferative Neoplasms Research and Treatment). Blood. 2010 Mar. 4; 115(9):1703-8). Variables included in the IPSS are age >65 years, constitutional symptoms, hemoglobin level <10 g/dL, and white blood cell (WBC) counts. Additional recent prognostic scoring systems include the Dynamic International Prognostic Scoring System Plus (DIPSS Plus) and scoring systems incorporating data from mutation analyses. There is a strong association between overall survival for MF patients and the DIPSS risk category for patients with low, intermediate risk 1, intermediate risk 2, or high risk with median survival of 15.4, 6.5, 2.9, and 1.3 years, respectively (Tefferi A. Primary myelofibrosis: 2017 update on diagnosis, risk-stratification, and management. Am. J. Hematol. 2016 December; 91(12): 1262-1271).

Approximately 70% of individuals with MF are in the intermediate-2 or high-risk categories (Gangat N, Caramazza D, Vaidya R, George G, Begna K, Schwager S, et al. DIPSS plus: a refined Dynamic International Prognostic Scoring System for primary myelofibrosis that incorporates prognostic information from karyotype, platelet count, and transfusion status. J. Clin. Oncol. 2011 Feb. 1; 29(4):392-7), representing the greatest unmet medical need. Symptomatic enlargement of the spleen and liver, the necessity for RBC transfusions, cachexia, and the other MF-associated symptoms result in greatly compromised quality of life in these patients (Mesa R A, Camoriano J K, Geyer S M, Wu W, Kaufmann S H, Rivera C E, et al. A phase II trial of tipifarnib in myelofibrosis: primary, post-polycythemia vera and post-essential thrombocythemia. Leukemia. 2007 September; 21(9): 1964-70).

Allogeneic stem-cell transplantation (SCT) is currently the only treatment that can induce long-term remission in patients with MF. The average age at diagnosis of MF is 65 years; thus, the majority of patients are not eligible for SCT. Therefore, the treatment options are primarily symptom-oriented, to help mitigate the clinical presentation of anemia, splenomegaly, constitutional symptoms and less commonly increased levels of platelets, and WBCs. So far, none of these symptom-oriented treatments has shown an anti-clonal effect, although alleviation in spleen size and splenic discomfort, symptoms, and anemia have been shown (Vannucchi A M, Harrison C N. Emerging treatments for classical myeloproliferative neoplasms. Blood. 2017 Feb. 9; 129(6):693-703).

Fortunately, the understanding of MPNs and the molecular mechanisms of the disease have been expanding. In 2005, the JAK2V617F mutation was discovered and observed in approximately 50% to 60% of patients with PMF or ET and 90% to 95% of patients with PV. This discovery, along with the observation of other mutations in patients with MPNs found to activate the JAK/signal transducers and activators of transcription (STAT) pathway (JAK2 exon 12, myeloproliferative leukemia, and adaptor protein LNK) (Oh S T, Simonds E F, Jones C, Hale M B, Goltsev Y, Gibbs K D, Jr., et al. Novel mutations in the inhibitory adaptor protein LNK drive JAK-STAT signaling in patients with myeloproliferative neoplasms. Blood. 2010 Aug. 12; 116(6):988-92; Pikman Y, Lee B H, Mercher T, McDowell E, Ebert B L, Gozo M, et al. MPLW515L is a novel somatic activating mutation in myelofibrosis with myeloid metaplasia. PLoS Med. 2006 July; 3(7):e270; Scott L M, Tong W, Levine R L, Scott M A, Beer P A, Stratton M R, et al. JAK2 exon 12 mutations in polycythemia vera and idiopathic erythrocytosis. N. Engl. J. Med. 2007 Feb. 1; 356(5):459-68), has established dysregulation of the JAK signaling pathway as the major contributor to the pathogenesis of MPNs. It has also translated into the development of small-molecule JAK inhibitors.

The JAK1/2 inhibitor ruxolitinib is currently the only approved therapy for MF. Ruxolitinib is indicated for treatment of patients with intermediate or high-risk MPN-associated myelofibrosis (MF), including primary MF, post-polycythemia vera MF and post-essential thrombocythemia MF. The registration of ruxolitinib was based on 2 randomized, controlled studies (COMFORT-I and COMFORT-II) that compared ruxolitinib to placebo and to the best available therapy (BAT), respectively (Harrison C, Vannucchi A D. Ruxolitinib: a potent and selective Janus kinase 1 and 2 inhibitor in patients with myelofibrosis. An update for clinicians. Ther. Adv. Hematol. 2012 December; 3(6):341-54; Verstovsek S, Mesa R A, Gotlib J, Levy R S, Gupta V, DiPersio J F, et al. A double-blind, placebo-controlled trial of ruxolitinib for myelofibrosis. N. Engl. J. Med. 2012 Mar. 1; 366(9):799-807). The studies demonstrated benefit, with a higher proportion of subjects in the ruxolitinib arms exhibiting a ≥35% reduction in spleen volume as measured by magnetic resonance imaging (MRI) at 24 weeks in COMFORT-I (41.9% ruxolitinib versus 0.7% placebo) and at 48 weeks in COMFORT-II (28.5% ruxolitinib versus 0% BAT). In COMFORT-I, there was a >50% improvement in the Myelofibrosis Symptom Assessment Form (MFSAF) Total Symptom Score (TSS) at 24 weeks in 45.9% of subjects on ruxolitinib compared with 5.3% of subjects on placebo. Improvement of survival in the ruxolitinib arm as compared with BAT was also demonstrated based on the recent 3-year follow-up data from the COMFORT-II study. The Kaplan-Meier estimated probability of survival at 144 weeks was 81% in the ruxolitinib arm and 61% in the BAT arm (Cervantes F, Kiladjian J J, Niederwieser D, Sirulnik A, Stalbovskaya V, McQuity M, et al. Long-Term Safety, Efficacy, and Survival Findings From Comfort-II, a Phase 3 Study Comparing Ruxolitinib with Best Available Therapy (BAT) for the Treatment of Myelofibrosis (MF). Blood. 2012; 120(21):801). Improvement in bone marrow fibrosis was observed in 15% of subjects receiving ruxolitinib at 24 months compared with 5% of subjects who received BAT; however, the improvements declined by 48 months in the small number of subjects who were available for follow-up. It is unclear if any of the subjects who were included in this study achieved clinical resolution of their spleen and symptoms (Kvasnicka H D. WHO classification of myeloproliferative neoplasms (MPN): A critical update. Curr. Hematol. Malig. Rep. 2013 December; 8(4):333-41).

Ruxolitinib is approved in the United States (US) and in the European Union (EU) for the treatment of MPN-associated myelofibrosis.

In the US, ruxolitinib (Jakafi®) was approved by the Food and Drug Administration (FDA) in November 2011 for the treatment of patients with intermediate or high-risk myelofibrosis, including primary myelofibrosis, post-polycythemia vera myelofibrosis and post-essential thrombocythemia myelofibrosis. Ruxolitinib has also been approved to treat polycythemia vera in patients who have had an inadequate response to or are intolerant to hydroxyurea.

In the EU, ruxolitinib (Jakavi®) was approved by the European Medicines Agency (EMA) in August 2012 for the treatment of disease-related splenomegaly or symptoms in adult patients with primary myelofibrosis (also known as chronic idiopathic myelofibrosis), post-polycythemia vera myelofibrosis or post-essential thrombocythemia myelofibrosis.

MPN-associated myelofibrosis, particularly intermediate or high-risk disease, is a serious and fatal condition. While the benefits of the ruxolitinib therapy in terms of spleen response and improvement of constitutional symptoms are significant, ruxolitinib is also associated with the risks of treatment-associated anemia (40.4% vs 12.3 for BAT) and thrombocytopenia (44.5% vs 9.65 for BAT) (Harrison C, Vannucchi A D. Ruxolitinib: a potent and selective Janus kinase 1 and 2 inhibitor in patients with myelofibrosis. An update for clinicians. Ther. Adv. Hematol. 2012 December; 3(6):341-54). The 1-, 2-, and 3-year discontinuation rates are 49, 71 and 86%, respectively. Major reasons for discontinuation are loss of therapeutic effect, lack of response and drug-induced cytopenias (Tefferi A, Pardanani A. JAK inhibitors in myeloproliferative neoplasms: rationale, current data and perspective. Blood Rev. 2011 September; 25(5):229-37). Additionally, responses to ruxolitinib are typically observed within the first 3-6 months after therapy initiation (Verstovsek S, Mesa R A, Gotlib J, Levy R S, Gupta V, DiPersio J F, et al. A double-blind, placebo-controlled trial of ruxolitinib for myelofibrosis. N. Engl. J. Med. 2012 Mar. 1; 366(9):799-807; Harrison C, Vannucchi A M. Ruxolitinib: a potent and selective Janus kinase 1 and 2 inhibitor in patients with myelofibrosis. An update for clinicians. Ther. Adv. Hematol. 2012 December; 3(6):341-54) and it has been suggested that for patients who have not had a reduction in spleen size or improvement in symptoms after that period, alternative therapies should be considered (Keohane C, Radia D H, Harrison C N. Treatment and management of myelofibrosis in the era of JAK inhibitors. Biologics. 2013; 7:189-98; Harrison C N, Mesa R A, Jamieson C, Hood J, Bykowski J, Zuccoli G, et al. Case Series of Potential Wernicke's Encephalopathy in Patients Treated with Fedratinib. Blood. 2017b; 130(Suppl 1), 4197. Accessed Mar. 23, 2018. Retrieved from http://www.bloodjournal.org/content/130/Suppl_1/4197). The effect of ruxolitinib on overall survival (OS) continues to be debated and the unclear limited effects on bone marrow fibrosis and driver mutation allele burden suggest that the disease-modifying activity of the drug is likely to be minor. Therefore, the unmet medical need for frontline myelofibrosis patients remains high, despite the availability of ruxolitinib, especially for patients who have low baseline platelet counts and are susceptible to myelosuppression/thrombocytopenia.

For patients who have been previously treated with a JAK inhibitor, there is no approved therapy and the prognosis is poor (Newberry K J, Patel K, Masarova L, Luthra R, Manshouri T, Jabbour E, et al. Clonal evolution and outcomes in myelofibrosis after ruxolitinib discontinuation. Blood. 2017 Aug. 31; 130(9):1125-31). Mechanisms of resistance to ruxolitinib remain unclear. It has been shown preclinically that myelofibrosis is intrinsically more resistant to JAK2 inhibition than polycythemia vera or essential thrombocythemia and there continues to be a major unmet need for a JAK2 inhibitor that is effective after ruxolitinib treatment failure. Moreover, the median survival of relapsed and refractory patients who discontinued ruxolitinib has been reported to be 6 months (Jabbour E, Hagop M, Kantarjian H M, Garcia-Manero G, Quintas-Cardama A, Cardena-Turanzas M, et al. Outcome of Patients (pts) With Myelofibrosis (MF) After Ruxolutinib (Rux) Therapy. Blood. 2013; 122(21):1584. Accessed Mar. 25, 2018. Retrieved from http://www.bloodjournal.org/content/122/21/1584). Notably, after a median follow-up of 10 months from stopping ruxolitinib, only 27 (34%) patients remained alive (Kantarjian H M, Silver R T, Komrokji R S, Mesa R A, Tacke R, Harrison C N. Ruxolitinib for myelofibrosis—an update of its clinical effects. Clin. Lymphoma Myeloma Leuk. 2013 December; 13(6):638-45). Only 27% of patients remained on therapy after 5 years in the COMFORT-I trial. Outcomes for patients who discontinue ruxolitinib in this situation are poor and such patients have bad outcomes.

Two randomized studies assessed the use of JAK inhibitors for subjects with previous ruxolitinib treatment in comparison to BAT that included continuous or retreatment with ruxolitinib.

In the PERSIST-2 study subjects with a platelet count 100×10⁹/L or less were randomized to receive either pacritinib 400 mg once daily, 200 mg twice daily or BAT including ruxolitinib. In the subgroup of subjects (n=95) with prior ruxolitinib, a spleen volume response of 35% or more at week 24 was achieved at week 24 in 2 subjects (6%) and 4 subjects (13%) and in 1 subject on BAT (3%) respectively (Mascarenhas J, Hoffman R, Talpaz M, Gerds A T, Stein B, Gupta V, et al. Pacritinib vs Best Available Therapy, Including Ruxolitinib, in Patients With Myelofibrosis: A Randomized Clinical Trial. JAMA Oncol. 2018 May 1; 4(5):652-9).

In the SIMPLFY-2 study MF subjects (n=156) with previous ruxolitinib treatment for at least 28 days who either required red blood cell transfusions while on ruxolitinib or ruxolitinib dose reduction to less than 20 mg twice a day with at least one of grade 3 thrombocytopenia, anemia, or bleeding at grade 3 or worse were randomized 2:1 to receive either momelotinib or BAT including ruxolitinib.

A spleen volume response of 35% or more at week 24 was achieved at week 24 in 7 subjects (7%) and 3 subjects on BAT (6%) (Harrison C N, Vannucchi A M, Platzbecker U, Cervantes F, Gupta V, Lavie D, et al. Momelotinib versus best available therapy in patients with myelofibrosis previously treated with ruxolitinib (SIMPLIFY 2): a randomised, open-label, phase 3 trial. Lancet Haematol. 2018 February; 5(2):e73-e81).

Currently, best available therapy (BAT) to treat patients with MF that have been previously treated ruxolitinib is limited. BAT may include retreatment with ruxolitinib, chemotherapy (e.g., hydroxyurea), anagrelide, corticosteroids, hematopoietic growth factor, immunomodulating agents, androgens, interferon, and may also include “no treatment” and symptom directed treatment.

Therefore, there remains an unmet need for the patients who have been previously treated with a JAK inhibitor due to the low life expectancy, notably high discontinuation rate with ruxolitinib and the myelosuppression.

Compound I

The synthesis of the Compound I is disclosed in Example 90 of U.S. Pat. No. 7,528,143, issued May 5, 2009, which is hereby incorporated by reference in its entirety. Compound I, also known as fedratinib, is a potent and selective inhibitor of JAK2 kinase activity that in cellular assays inhibits JAK2 signaling, cellular proliferation driven by mutant JAK2 or mutant MPL, and induces apoptosis in cells expressing constitutively active JAK2. Compound I also inhibits erythroid colony formation of hematopoietic progenitors isolated from myeloproliferative neoplasm (MPN) patients.

Eighteen clinical studies were conducted with fedratinib. Fedratinib has been studied extensively in the treatment of patients with MPN-associated myelofibrosis.

Fedratinib demonstrated clinical efficacy in a randomized, placebo-controlled, Phase 3 study (JAKARTA [EFC12153]) in patients with intermediate-2 or high-risk MF who were previously untreated. The primary endpoint was response rate, defined as the proportion of subjects who had a ≥35% reduction in spleen volume from baseline to the End of Cycle 6 and confirmed 4 weeks later by MRI. Analyses for spleen response were also performed at the end of Cycle 6 (e.g., regardless of confirmation), as recommended by the IWG-MRT Criteria. Symptom response rate (SRR), based on a patient-reported outcome (PRO) tool, the modified Myelofibrosis Symptom Assessment Form (MFSAF) that assessed 6 key MF-associated symptoms (night sweats, pruritus, abdominal discomfort, early satiety, pain under ribs on left side, and bone or muscle pain) was a key secondary endpoint. The SRR was defined as the proportion of subjects with a ≥50% reduction in the Total Symptom Score (TSS) of the modified MFSAF diary from baseline to the end of Cycle 6. Both endpoints are measures for demonstrating clinical benefit in the proposed population. The response rate per primary endpoint was 36.5% and 40.2% at the 400 mg (proposed dose for this study) and 500 mg daily dose respectively vs 1% on the placebo arm. The response rate at Cycle 6 as recommended by IWG-MRT was of 46.9% and 49.5% in patients treated with the 400 mg and 500 mg daily doses respectively. A total of 36.3% and 34.1% of subjects at the dose of 400 mg and 500 mg doses respectively achieved a ≥50% reduction in TSS compared with 7.1% of subjects receiving placebo. Median duration of response (≥35% reduction in spleen volume) was 10.4 months for responders from both active groups (400-mg and 500-mg groups). The most common treatment-emergent adverse events (TEAEs) of all grades reported in the fedratinib 400 mg daily dose group were diarrhea 65.6%, nausea 63.5%, anemia (G3 and G4) 42.7%, vomiting 41.7%, fatigue 15.6%, and peripheral edema 15.6%. The 400 mg dose was confirmed to be better tolerated than the 500 mg dose, in particular with fewer subjects reporting Grade 3 or 4 TEAEs (70.8% and 78.4%, respectively), treatment-emergent serious adverse events (SAEs) (38.5% and 44.3%, respectively) and TEAEs leading to permanent treatment discontinuation (27.1% and 36.1%, respectively) (Pardanani A, Tefferi A, Jamieson C, Gabrail N Y, Lebedinsky C, Gao G, et al. A phase 2 randomized dose-ranging study of the JAK2-selective inhibitor fedratinib (SAR302503) in patients with myelofibrosis. Blood Cancer J. 2015 Aug. 7; 5:e335).

The single-arm Phase 2 JAKARTA2 study (ARD12181) enrolled patients with intermediate-1 with symptoms, intermediate-2 or high-risk MPN-associated myelofibrosis who have been previously treated with ruxolitinib. The primary endpoint was response rate, which was defined as the proportion of subjects who have a ≥35% reduction from baseline in spleen volume to the End of Cycle 6 in the per protocol defined population.

As in the Phase 3 JAKARTA study, one of the key secondary endpoints was symptom response rate (SRR), defined as the proportion of subjects with a ≥50% reduction in the TSS using the modified MFSAF diary from baseline to the end of Cycle 6.

Resistance to ruxolitinib was defined as any one of the following: a) Lack of response (absence of response); b) disease progression (spleen size increase during ruxolitinib treatment); or c) loss of response at any time during ruxolitinib treatment. Intolerance to ruxolitinib was defined as any one of the following: a) hematologic toxicity (anemia, thrombocytopenia, others); b) non-hematologic toxicity.

The overall spleen response rate (proportion of patients with ≥35% reduction from baseline in spleen volume to the End of Cycle 6) was 55.4%. A total of 25.6% of subjects achieved a ≥50% reduction in TSS.

All 97 patients had at least 1 TEAE (all grades); Grade 3 or 4 TEAEs were reported by 62.9% patients. The most common nonhematologic TEAEs (reported by ≥10% of patients) (all grades) were gastrointestinal disorders including diarrhea (61.9%), nausea (55.7%), and vomiting (41.2%). The most common hematologic TEAEs (reported by >10 patients) (all grades) were anemia (48.5%) and thrombocytopenia (26.8%). Thirty-eight and one-tenth percent experienced Grade 3 or 4 anemia and 21.6% experienced Grade 3 or 4 thrombocytopenia. No grade 5 hematologic TEAEs were reported. Anti-infectives for systemic use were given to 55.7% patients in the study (Harrison C N, McLornan D P. Current treatment algorithm for the management of patients with myelofibrosis, JAK inhibitors, and beyond. Hematology Am. Soc. Hematol. Educ. Program. 2017 Dec. 8; 2017(1):489-97).

Myelofibrosis is a clonal disease resulting from mutations in hematopoietic stem cells that promote abnormal proliferation and myeloid differentiation (Mead A J, Mullally A. Myeloproliferative neoplasm stem cells. Blood. 2017 Mar. 23; 129(12):1607-16). In addition to JAK2V617F, several other mutations, in JAK2 and other genes, are found in MF patients and have been associated with prognosis, AML progression, and response to the JAK inhibitor ruxolitinib (Vainchenker W, Kralovics R. Genetic basis and molecular pathophysiology of classical myeloproliferative neoplasms. Blood. 2017 Feb. 9; 129(6):667-679, Tefferi A, Guglielmelli P, Nicolosi M, Mannelli F, et al. GIPSS: genetically inspired prognostic scoring system for primary myelofibrosis. Leukemia. 2018 Mar. 23; Spiegel J Y, McNamara C, Kennedy J A, Panzarella T, et al. Impact of genomic alterations on outcomes in myelofibrosis patients undergoing JAK1/2 inhibitor therapy. Blood. 2017 Sep. 8; 1(20):1729-1738; Newberry K J, Patel K, Masarova L, Luthra R, et al. Clonal evolution and outcomes in myelofibrosis after ruxolitinib discontinuation. Blood. 2017 Aug. 31; 130(9):1125-1131; Patel K P, Newberry K J, Luthra R, Jabbour E, et al. Correlation of mutation profile and response in patients with myelofibrosis treated with ruxolitinib. Blood. 2015 Aug. 6; 126(6):790-7; Levine R L, Wadleigh M, Cools J, Ebert B L, Wernig G, Huntly B J, et al. Activating mutation in the tyrosine kinase JAK2 in polycythemia vera, essential thrombocythemia, and myeloid metaplasia with myelofibrosis. Cancer Cell. 2005; 7:387-97; Werning G, Mercher T, Okabe R, Levine L, Lee B H, Gilliland G L. Expression of JAK2V617F causes a polycythemia vera-like disease with associated myelofibrosis in a murine bone marrow transplant model. Blood. 2006; 107:4274-81; Mercher T, Wernig G, Moore S A, Levine R L, Gu T L, Frohling S, Cullen D, Polakiewicz R D, Bernard O A, Boggon T J, Lee B H, Gilliland D G. JAK2T875N is a novel activating mutation that results in myeloproliferative disease with features of megakaryoblastic leukemia in a murine bone marrow transplantation model. Blood. 2006 Oct. 15; 108(8):2770-9; Scott L M, Tong W, Levine R L, Scott M A, Beer P A, Stratton M R, et al. JAK2 exon 12 mutations in polycythemia vera and idiopathic erythrocytosis. N. Engl. J. Med. 2007 Feb. 1; 356(5):459-68; Pardanani A, Tefferi A, Jamieson C, Gabrail N Y, et al. A phase 2 randomized dose-ranging study of the JAK2-selective inhibitor fedratinib (SAR302503) in patients with myelofibrosis. Blood Cancer J. 2015 Aug. 7; 5:e335). Fedratinib was reported to decrease JAK2V617F frequency in non-clinical models of MF (Wernig G, Kharas M G, Okabe R, Moore S A, Leeman D S, Cullen D E, et al. Efficacy of TG101348, a selective JAK2 inhibitor, in treatment of a murine model of JAK2V617F-induced polycythemia vera. Cancer Cell. 2008 April; 13(4):311-20) and in MF patients (Pardanani A, Gotlib J R, Jamieson C, Cortes J E, Talpaz M, Stone R M, et al. Safety and efficacy of TG101348, a selective JAK2 inhibitor, in myelofibrosis. J. Clin. Oncol. 2011 Mar. 1; 29(7):789-96).

Abnormal cytokine expression and bone marrow fibrosis are hallmarks of MF (Vainchenker W, Kralovics R. Genetic basis and molecular pathophysiology of classical myeloproliferative neoplasms. Blood. 2017 Feb. 9; 129(6):667-679; Mondet J, Hussein K, Mossuz P. Circulating Cytokine Levels as Markers of Inflammation in Philadelphia Negative Myeloproliferative Neoplasms: Diagnostic and Prognostic Interest. Mediators Inflamm. 2015:670580). High levels of pro-inflammatory and fibrogenic cytokines have been reported to contribute to bone marrow (BM) stromal changes, ineffective erythropoiesis/extramedullary hematopoiesis and constitutional symptoms in MF (Mondet J, Hussein K, Mossuz P. Circulating Cytokine Levels as Markers of Inflammation in Philadelphia Negative Myeloproliferative Neoplasms: Diagnostic and Prognostic Interest. Mediators Inflamm. 2015:670580; Tefferi A, Pardanani A. JAK inhibitors in myeloproliferative neoplasms: rationale, current data and perspective. Blood Rev. 2011 September; 25(5):229-37). Fedratinib was found to modulate circulating cytokines in MF patients not previously treated with JAK inhibitors (Pardanani A, Tefferi A, Jamieson C, Gabrail N Y, et al. A phase 2 randomized dose-ranging study of the JAK2-selective inhibitor fedratinib (SAR302503) in patients with myelofibrosis. Blood Cancer J. 2015 Aug. 7; 5:e335). Cytokine modulation correlated with sustained viral response and improvement in constitutional symptoms in these patients (Pardanani A, Tefferi A, Jamieson C, Gabrail N Y, et al. A phase 2 randomized dose-ranging study of the JAK2-selective inhibitor fedratinib (SAR302503) in patients with myelofibrosis. Blood Cancer J. 2015 Aug. 7; 5:e335). However, the effect of fedratinib on circulating cytokines in patients previously exposed to ruxolitinib has not been characterized.

Recent studies are starting to unveil immune regulatory roles for JAK2V617F, as well as for JAK inhibitors like ruxolitinib and fedratinib. For instance, JAK2V617F was reported to contribute to immune evasion of MPN myeloid cells by upregulation of program death-ligand 1 (PD-L1) (Prestipino A, Emhardt A J, Aumann K, O'Sullivan D, et. al. Oncogenic JAK2V617F causes PD-L1 expression, mediating immune escape in myeloproliferative neoplasms. Sci. Transl. Med. 2018 Feb. 21; 10(429)). Ruxolitinib has been reported to modulate PD-L1 expression in these cells (Prestipino A, Emhardt A J, Aumann K, O'Sullivan D, et. al. Oncogenic JAK2V617F causes PD-L1 expression, mediating immune escape in myeloproliferative neoplasms. Sci. Transl. Med. 2018 Feb. 21; 10(429)). Fedratinib was reported to modulate PD-L1 expression in lymphoma tumor cells (Hao Y, Chapuy B, Monti S, Sun H H, Rodig S J, Shipp M A. Selective JAK2 inhibition specifically decreases Hodgkin lymphoma and mediastinal large B-cell lymphoma growth in vitro and in vivo. Clin Cancer Res. 2014; 20(10):2674-83). Preclinical and clinical data indicate that ruxolitinib can act a potent immunosuppressive drug, suppressing graft-versus-host disease (GVHD), decreasing frequencies and impairing activation of T- and NK-cells of MF patients (Betts B C, Bastian D, Iamsawat S, Nguyen H, et al. Targeting JAK2 reduces GVHD and xenograft rejection through regulation of T cell differentiation. Proc Natl Acad Sci USA. 2018 Feb. 13; 115(7):1582-1587. Epub 2018; Schonberg K, Rudolph J, Vonnahme M, Parampalli et al. JAK Inhibition Impairs NK Cell Function in Myeloproliferative Neoplasms. Cancer Res. 2015 Jun. 1; 75(11):2187-99; Parampalli Yajnanarayana S, Stubig T, Cornez I, Alchalby H, et al. JAK1/2 inhibition impairs T cell function in vitro and in patients with myeloproliferative neoplasms. Br. J. Haematol. 2015 June; 169(6):824-33). Preclinical data suggest that fedratinib is able to modulate PD-L1 expression in lymphoma tumor cells (Hao Y, Chapuy B, Monti S, Sun H H, Rodig S J, Shipp M A. Selective JAK2 inhibition specifically decreases Hodgkin lymphoma and mediastinal large B-cell lymphoma growth in vitro and in vivo. Clin. Cancer Res. 2014 May 15; 20(10):2674-83). However, non-clinical data indicates that fedratinib exerts weak effect on GVHD (Betts B C, Veerapathran A, Pidala J, Yang H, et al. Targeting Aurora kinase A and JAK2 prevents GVHD while maintaining Treg and antitumor CTL function. Sci. Transl. Med. 2017 Jan. 11; 9(372)), and T cell development (Wernig G, Kharas M G, Okabe R, Moore S A, Leeman D S, Cullen D E, et al. Efficacy of TG101348, a selective JAK2 inhibitor, in treatment of a murine model of JAK2V617F-induced polycythemia vera. Cancer Cell. 2008 April; 13(4):311-20).

Methods of Treating Myeloproliferative Disorders

In some embodiments, the present disclosure methods of treating, stabilizing or lessening the severity or progression of one or more myeloproliferative disorders. In certain embodiments, the present disclosure provides methods of treating a patient previously treated with ruxolitinib (JAKAFI®; (3R)-3-cyclopentyl-3-[4-(7H-pyrrolo[2,3-d]pyrimidin-4-yl)pyrazol-1-yl]propanenitrile).

In some embodiments, provided methods comprise administering to a patient previously treated with ruxolitinib Compound I:

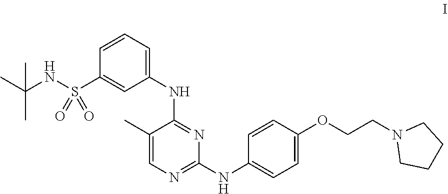 [SMILES: Cc1cnc(Nc2ccc(OCCN3CCCC3)cc2)nc1Nc1cccc(S(=O)(=O)NC(C)(C)C)c1]

or a pharmaceutically acceptable salt or hydrate thereof. In some embodiments, Compound I is in the form of a dihydrochloride salt. Compound I, or a pharmaceutically acceptable salt thereof, may also exist in a hydrate form. In some such embodiments, Compound I is in the form of a dihydrochloride monohydrate. Accordingly, in some embodiments, provided methods comprise administering to a patient in need thereof Compound II:

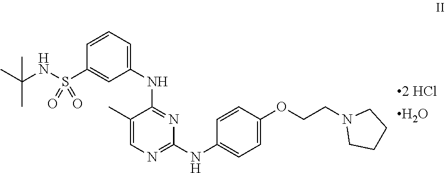

In some embodiments, the patient has been previously treated with ruxolitinib for at least 3 months. In some embodiments, the patient has been previously treated with ruxolitinib for at least 3 months with inadequate efficacy response defined as <10% spleen volume reduction by Mill. In some embodiments, the patient has been previously treated with ruxolitinib for at least 3 months with inadequate efficacy response defined as <30% decrease from baseline in spleen size by palpation. In some embodiments, the patient has experienced regrowth to <10% spleen volume reduction by MM following an initial response. In some embodiments, the patient has experienced regrowth to <30% decrease from baseline in spleen size by palpation following an initial response. Patients who experience inadequate efficiency are said to be refractory. Patients who experience regrowth to those parameters are said to be relapsed.

In some embodiments, the patient has been previously treated with ruxolitinib for at least 28 days complicated by
i. development of a red blood cell transfusion requirement; or
ii. Grade ≥3 adverse event(s) of thrombocytopenia, anemia, hematoma, and/or hemorrhage while on treatment with ruxolitinib.

In some embodiments, the patient is suffering from or has been diagnosed with a myeloproliferative disorder that is unresponsive to ruxolitinib.

In some embodiments, the patient is suffering from or has been diagnosed with a myeloproliferative disorder that is refractory or resistant to ruxolitinib.

In some embodiments, the patient has relapsed during or following ruxolitinib therapy.

In some embodiments, the patient is intolerant to ruxolitinib. In some embodiments, patient intolerance to ruxolitinib is evidenced by a hematological toxicity (e.g., anemia, thrombocytopenia, etc.) or a non-hematological toxicity.

In some embodiments, the patient has had an inadequate response to or is intolerant to hydroxyurea.

In some embodiments, the patient is exhibiting or experiencing, or has exhibited or experienced, one or more of the following during treatment with ruxolitinib: lack of response, disease progression, or loss of response at any time during ruxolitinib treatment. In some embodiments, disease progression is evidenced by an increase in spleen size during ruxolitinib treatment.

In some embodiments, a patient previously treated with ruxolitinib has a somatic mutation or clonal marker associated with or indicative of a myeloproliferative disorder. In some embodiments, the somatic mutation is selected from a JAK2 mutation, a CALR mutation or a MPL mutation. In some embodiments, the JAK2 mutation is V617F. In some embodiments, the CALR mutation is a mutation in exon 9. In some embodiments, the MPL mutation is selected from W515K and W515L.

In some embodiments, the present disclosure provides a method of treating a relapsed or refractory myeloproliferative disorder, wherein the myeloproliferative disorder is relapsed or refractory to ruxolitinib.

In some embodiments, a myeloproliferative disorder is selected from intermediate risk MPN-associated myelofibrosis and high risk MPN-associated myelofibrosis.

In some embodiments, the intermediate risk MPN-associated myelofibrosis is selected from primary myelofibrosis, post-polycythemia vera (post-PV) myelofibrosis and post-essential thrombocythemia (post-ET) myelofibrosis. In some embodiments, the MPN-associated myelofibrosis is intermediate risk 1 (also referred to as intermediate-1 risk). In some embodiments, the MPN-associated myelofibrosis is intermediate risk 2 (also referred to as intermediate-2 risk).

In some embodiments, the high risk MPN-associated myelofibrosis is selected from primary myelofibrosis, post-polycythemia vera (post-PV) myelofibrosis and post-essential thrombocythemia (post-ET) myelofibrosis.

In some embodiments, the present disclosure provides a method of reducing spleen volume by at least 25% in a patient suffering from or diagnosed with a myeloproliferative disorder. In some embodiments, the patient's spleen volume is reduced by at least 35%. In some embodiments, spleen volume is measured by magnetic resonance imaging (MRI), computed tomography (CT) and/or palpation. In some embodiments, the at least 35% reduction in spleen volume occurs by the end of cycle 6.

In some embodiments, the present disclosure provides a method of improving overall survival in a patient suffering from or diagnosed with a myeloproliferative disorder. In some embodiments, the overall survival is improved relative to best available therapy.

In some embodiments, the present disclosure provides a method of improving symptom response rate in a patient suffering from or diagnosed with a myeloproliferative disorder. In some such embodiments, symptom response rate is evidenced by at least 50% reduction in total symptom score (TSS). In some embodiments, symptom response rate is evidenced by at least 50% reduction in total symptom score (TSS) at 48 weeks. In some embodiments, symptom response rate is evidenced by at least 50% reduction in total symptom score (TSS) at 24 weeks. In some embodiments, the symptom response rate is improved relative to best available therapy.

In some embodiments, the present disclosure provides a method of increasing the median survival in a patient population that has relapsed or is refractory to ruxolitinib. In some embodiments, the median survival in patients who have relapsed or are refractory to ruxolitinib is greater than 6 months. In some embodiments, the median survival in patients who have relapsed or are refractory to ruxolitinib is greater than 1 year. In some embodiments, the median survival in patients who have relapsed or are refractory to ruxolitinib is greater than 1.5 years. In some embodiments, the median survival in patients who have relapsed or are refractory to ruxolitinib is greater than 3 years. In some embodiments, the median survival in patients who have relapsed or are refractory to ruxolitinib is greater than 5 years. In some embodiments, the median survival is increased relative to best available therapy.

In some embodiments, the present disclosure provides a method of decreasing allele burden in a patient having a somatic mutation or clonal marker associated with or indicative of a myeloproliferative disorder. In some embodiments, the allele burden is decreased relative to the patient's allele burden prior to treatment with Compound I, or a pharmaceutically acceptable salt or hydrate thereof. In some embodiments, the somatic mutation is selected from a JAK2 mutation, a CALR mutation or a MPL mutation. In some embodiments, the JAK2 mutation is V617F. In some embodiments, the CALR mutation is a mutation in exon 9. In some embodiments, the MPL mutation is selected from W515K and W515L.

In some embodiments, provided methods induce a complete response (CR). In some embodiments, a complete response includes one or more of the following:
Bone marrow:* Age-adjusted normocellularity; <5% blasts; ≤grade 1 myelofibrosis and
Peripheral blood: Hemoglobin ≥100 g/L and <upper normal limit (UNL); neutrophil count ≥1×10⁹/L and <UNL;
Platelet count ≥100×10⁹/L and <UNL; <2% immature myeloid cells and
Clinical: Resolution of disease symptoms; spleen and liver not palpable; no evidence of extramedullary hematopoiesis (EMH)

In some embodiments, provided methods induce a partial response (PR). In some embodiments, a partial response includes one or more of the following:
Peripheral blood: Hemoglobin ≥100 g/L and <UNL; neutrophil count ≥1×10⁹/L and <UNL; platelet count ≥100×10⁹/L and <UNL; <2% immature myeloid cells and
Clinical: Resolution of disease symptoms; spleen and liver not palpable; no evidence of EMH, or
Bone marrow:* Age-adjusted normocellularity; <5% blasts; <grade 1 myelofibrosis, and peripheral blood: Hemoglobin ≥85 but <100 g/L and <UNL; neutrophil count ≥1×10⁹/L and <UNL; platelet count ≥50, but <100×10⁹/L and <UNL; <2% immature myeloid cells and
Clinical: Resolution of disease symptoms; spleen and liver not palpable; no evidence of EMH

In some embodiments, provided methods induce a clinical improvement (CI). In some embodiments, clinical improvement includes the achievement of anemia, spleen or symptoms response without progressive disease or increase in severity of anemia, thrombocytopenia, or neutropenia.

In some embodiments, provided methods induce a spleen response. In some embodiments, a spleen response includes one or more of the following:
A baseline splenomegaly that is palpable at 5-10 cm, below the left costal margin (LCM), becomes not palpable or
A baseline splenomegaly that is palpable at >10 cm, below the LCM, decreases by ≥50%
A baseline splenomegaly that is palpable at <5 cm, below the LCM, is not eligible for spleen response
A spleen response requires confirmation by MRI or computed tomography showing ≥35% spleen volume reduction

In some embodiments, provided methods induce spleen and disease progression free survival (SDPFS) as compared to best available therapy.

In some embodiments, the present disclosure provides a method of minimizing one or more adverse events relating to or resulting from treatment with Compound I and/or Compound II. In some embodiments, the patient is at risk for developing Wernicke's encephalopathy. In some such embodiments, the patient is monitored for Wernicke's encephalopathy.

In some embodiments, the myeloproliferative disorder is myelofibrosis. In some embodiments, the myelofibrosis is primary myelofibrosis. In some embodiments, the myelofibrosis is secondary myelofibrosis. In some embodiments, the myelofibrosis is post-essential thrombocythemia myelofibrosis. In some embodiments, the myelofibrosis is post-polycythemia vera myelofibrosis.

In some embodiments, the myeloproliferative disorder is polycythemia vera. In some embodiments, the myeloproliferative disorder is essential thrombocythemia. In some embodiments, the myeloproliferative disorder is acute myeloid leukemia.

In some embodiments, Compound I is administered in the form of a hydrochloride salt. In some such embodiments, Compound I is administered in the form of a dihydrochloride salt. In some embodiments, Compound I is administered in the form of a dihydrochloride monohydrate (e.g., Compound II). It will be understood that references to Compound I herein are intended to encompass all salts and forms, including the hydrochloride salt, the dihydrochloride salt and the dihydrochloride monohydrate form.

In some embodiments, Compound I, or a pharmaceutically acceptable salt or hydrate thereof, (e.g., Compound II), is administered to the patient in a unit dosage form. In some embodiments, the unit dosage form of Compound I or Compound II is the molar equivalent of the free base weight of the compound. For example, a 100 mg dose of the free base form of Compound I equates to about 117.30 mg of Compound I in its dihydrochloride monohydrate form (i.e., Compound II). In some embodiments, the unit dosage form of Compound I or Compound II is about 50 mg, about 100 mg, about 150 mg, or about 200 mg, wherein the amount of Compound I or Compound II is the molar equivalent of the free base weight of the compound. In some embodiments, the unit dosage form of Compound I or Compound II is 100 mg, wherein the amount of Compound II is the molar equivalent of the free base weight of the compound.

In some embodiments, Compound I, or a pharmaceutically acceptable salt or hydrate thereof, (e.g., Compound II), is administered in an oral dosage form. In some such embodiments, the oral dosage form is a capsule. In some embodiments, the oral dosage form is a tablet.

In some embodiments, Compound I, or a pharmaceutically acceptable salt or hydrate thereof, (e.g., Compound II), is administered once daily (QD). In some embodiments, Compound I, or a pharmaceutically acceptable salt or hydrate thereof, (e.g., Compound II), is administered at a total daily dose of about 200 mg, about 300 mg or about 400 mg. In some embodiments, Compound I or Compound II is administered to the patient at a total daily dose of about 400 mg. In some embodiments, Compound I or Compound II is administered to the patient at a total daily dose of about 300 mg. In some embodiments, Compound I or Compound II is administered to the patient at a total daily dose of about 200 mg. In some embodiments, the total daily dose of Compound I or Compound II is modified due to an adverse event. In some embodiments, the total daily dose of Compound I or Compound II is reduced. In some embodiments, the total daily dose of Compound I or Compound II is reduced from about 400 mg to about 300 mg. In some embodiments, the total daily dose of Compound I or Compound II is reduced to about 200 mg. It will be appreciated that the amount (e.g., total daily dose) of Compound I or Compound II is the molar equivalent to, e.g., about 400 mg, about 300 mg or about 200 mg of the free base weight.

In some embodiments, Compound I, or a pharmaceutically acceptable salt or hydrate thereof, (e.g., Compound II), is administered once daily for a 28-day cycle. In some embodiments, Compound I, or a pharmaceutically acceptable salt or hydrate thereof, (e.g., Compound II), is administered once daily for two 28-day cycles. In some embodiments, Compound I, or a pharmaceutically acceptable salt or hydrate thereof, (e.g., Compound II), is administered once daily for three, four, five, or more 28-day cycles. In some embodiments, Compound I, or a pharmaceutically acceptable salt or hydrate thereof, (e.g., Compound II), is administered once daily for six, seven, eight, nine, ten, eleven, twelve or more 28-day cycles. In some embodiments, Compound I, or a pharmaceutically acceptable salt or hydrate thereof, (e.g., Compound II), is administered once a day for at least six 28-day cycles. In some embodiments, Compound I, or a pharmaceutically acceptable salt or hydrate thereof, (e.g., Compound II), is administered once daily until symptoms of disease are no longer measureable. In some embodiments, Compound I or Compound II is administered for the duration of a patient's life. In some embodiments, Compound I or Compound II is administered once daily for one or more 28-day cycles, followed by a dose holiday. A “dose holiday” as used herein refers to a period of time wherein Compound I or Compound II is not administered to the patient. In some embodiments, a dose holiday is one day, one week, or one 28-day cycle. In some embodiments, Compound I or Compound II is administered once daily for one or more 28-day cycles, followed by a dose holiday, and then resumption of administration of Compound I or Compound II once daily at the same dose level prior to the dose holiday. In some embodiments, Compound I or Compound II is administered once daily for one or more 28-day cycles, followed by a dose holiday, and then resumption of administration of Compound I or Compound II once daily at a dose level that is 100 mg less than the dose of Compound I or Compound II prior to the dose holiday. In some embodiments, the total daily dose of Compound I or Compound II is titrated upward by 100 mg following a prior dose reduction. It will be appreciated that the amount (e.g., total daily dose) of Compound I or Compound II is the molar equivalent to, e.g., about 400 mg, about 300 mg or about 200 mg of the free base weight.

In some embodiments, the patient has a myeloproliferative disease or condition. In some embodiments, the myeloproliferative disease or condition is selected from primary myelofibrosis, secondary myelofibrosis, polycythemia vera, and essential thrombocythemia. In some embodiments, secondary myelofibrosis is selected from post-polycythemia vera myelofibrosis and post-essential thrombocythemia myelofibrosis. In some embodiments, the myeloproliferative disorder is acute myeloid leukemia (AML). In some embodiments, the primary myelofibrosis is Dynamic International Prognostic Scoring System (DIPSS) intermediate or high-risk primary myelofibrosis. In some embodiments, said method comprises administering to a patient in need thereof a composition comprising Compound I, or a pharmaceutically acceptable salt or hydrate thereof. In some embodiments, provided methods comprise administering to a patient in need thereof a composition comprising Compound II.

In some embodiments, the previous therapy is a treatment with Compound I, or a pharmaceutically acceptable salt thereof or a hydrate thereof. In some embodiments, the previous therapy has been discontinued upon indication of elevated levels of amylase, lipase, aspartate aminotransferase (“AST”), alanine aminotransferase (“ALT”), and/or creatinine. In some embodiments, the previous therapy has been discontinued upon indication of a hematologic condition selected from the group consisting of anemia, thrombocytopenia, and neutropenia.

Each of the references listed herein is hereby incorporated by reference in its entirety.

### EXEMPLIFICATION

### Example 1

Protocol Summary. The study will enroll approximately 192 subjects randomized 2:1 to one of two arms in a multicenter, open-label, randomized, multinational study in subjects previously treated with ruxolitinib and with DIPSS (Dynamic International Prognostic Scoring System) intermediate or high-risk primary myelofibrosis (PMF), post-polycythemia vera myelofibrosis (post-PV MF), or post-essential thrombocythemia myelofibrosis (post-ET MF).

Objectives. The primary objective of the study is to evaluate percentage of subjects with at least 35% spleen volume reduction in the fedratinib and the best available therapy (BAT) arms. The secondary objectives are:
To evaluate myelofibrosis (MF)-associated symptoms as measured by the Myelofibrosis Symptom Assessment Form (MFSAF)
To evaluate the percentage of subjects with at least 25% spleen volume reduction (SVR)
To evaluate the safety of fedratinib
To evaluate the reduction of spleen size by palpation
To evaluate durability of spleen response by MRI/CT and by palpation
To evaluate the durability of symptoms response
To evaluate spleen and disease progression free survival
To assess the effectiveness of the risk mitigation strategy for gastrointestinal events and Wernicke encephalopathy (WE)
To evaluate Health-Related Quality of Life (HRQoL) as measured by the European Organization for Research and Treatment of Cancer Quality of Life C30 (EORTC QLQ-C30)
To evaluate Patient Reported Outcomes (PRO) as measured by the EQ-5D-5L questionnaire
To evaluate Overall Survival (OS)

The exploratory objectives are:
To evaluate time to spleen response by palpation
To evaluate spleen response by MM-CT with the best response during the first 6 cycles
To explore pharmacodynamic effects (e.g., circulating cytokines, hematopoietic cell profiling) of fedratinib activity in relation to efficacy parameters
To explore prognostic markers (e.g., gene mutations) in relation to efficacy parameters
To evaluate population pharmacokinetics and exposure-response relationship of fedratinib for subjects receiving fedratinib treatment
To assess the effect of study treatment on selected treatment-related symptoms from the subject's perspective (diarrhea, nausea, vomiting, dizziness, headache), assessed by the Patient Reported Outcome Version of the Common Terminology Criteria for Adverse Events (PRO-CTCAE).

Study Population. The approximately 192 subjects will be randomized 2:1 in the fedratinib arm or the best available therapy (BAT) arm.

Stratification at Randomization According to:
Risk category (DIPSS) Int-1 and Int-2 versus High Risk
Spleen size by palpitation: <15 cm below LCM versus ≥15 cm below LCM
Platelets 100,000/μL versus platelets <100,000/μL

Inclusion Criteria. Subjects must satisfy the following criteria to be enrolled in the study:
1. Subject is at least 18 years of age at the time of signing the informed consent form (ICF)
2. Subject has an Eastern Cooperative Oncology Group (ECOG) Performance Score (PS) of 0, 1 or 2
3. Subject has diagnosis of primary myelofibrosis (PMF) according to the 2016 World Health Organization (WHO) criteria, or diagnosis of post-ET or post-PV myelofibrosis according to the IWG-MRT 2007 criteria, confirmed by the most recent local pathology report
4. Subject has a DIPSS Risk score of Intermediate or High
5. Subject has a measurable splenomegaly during the screening period as demonstrated by spleen volume of ≥450 cm³ by MRI or CT-scan assessment or by palpable spleen measuring ≥5 cm below the left costal margin
6. Subject has been previously exposed to ruxolitinib, and must meet at least one of the following criteria (a or b)
   a. Treatment with ruxolitinib for ≥3 months with inadequate efficacy response defined as <10% spleen volume reduction by MRI or <30% decrease from baseline in spleen size by palpation or regrowth to these parameters following an initial response
   b. Treatment with ruxolitinib for ≥28 days complicated by any of the following:
      Development of a red blood cell transfusion requirement (at least 2 units/month for 2 months) or
      Grade ≥3 AEs of thrombocytopenia, anemia, hematoma, and/or hemorrhage while on treatment with ruxolitinib
7. Subject must have treatment-related toxicities from prior therapy resolved to Grade 1 or pretreatment baseline before start of last therapy prior to randomization
8. Subject must understand and voluntarily sign an ICF prior to any study-related assessments/procedures being conducted
9. Subject is willing and able to adhere to the study visit schedule and other protocol requirements
10. A female of childbearing potential (FCBP) must:
   a. Have two negative pregnancy tests as verified by the Investigator during screening prior to starting study therapy. She must agree to ongoing pregnancy testing during the course of the study, and after end of study treatment. This applies even if the subject practices true abstinence* from heterosexual contact.
   b. Either commit to true abstinence* from heterosexual contact (which must be reviewed on a monthly basis and source documented) or agree to use, and be able to comply with acceptable effective contraception** without interruption, −14 days prior to starting investigational product, during the study therapy (including dose interruptions), and for 28 days after discontinuation of study therapy. ** Agreement to use highly effective methods of contraception that alone or in combination resulting in a failure rate of a Pearl index of less than 1% per year when used consistently and correctly throughout the course of the study. Such methods include: Combined (estrogen and progestogen containing) hormonal contraception: Oral; Intravaginal; Transdermal; Progestogen-only hormonal contraception associated with inhibition of ovulation: Oral; Injectable hormonal contraception; Implantable hormonal contraception; Placement of an intrauterine device (IUD); Placement of an intrauterine hormone-releasing system (IUS); Bilateral tubal occlusion; Vasectomized partner.
Note: A female of childbearing potential (FCBP) is a female who: 1) has achieved menarche at some point, 2) has not undergone a hysterectomy or bilateral oophorectomy, or 3) has not been naturally postmenopausal (amenorrhea following cancer therapy does not rule out childbearing potential) for at least 24 consecutive months (i.e. has had menses at any time in the preceding 24 consecutive months).
11. A male subject must:
Practice true abstinence* (which must be reviewed on a monthly basis) or agree to use a condom during sexual contact with a pregnant female or a female of childbearing potential while participating in the study, during dose interruptions and for at least 30 days following investigational product discontinuation, or longer if required for each compound and/or by local regulations, even if he has undergone a successful vasectomy. * True abstinence is acceptable when this is in line with the preferred and usual lifestyle of the subject. [Periodic abstinence (eg, calendar, ovulation, symptothermal, post-ovulation methods) and withdrawal are not acceptable methods of contraception].

Exclusion Criteria. The presence of any of the following will exclude a subject from enrollment:
1. Any of the following laboratory abnormalities:
   a. Platelets <50,000/μL
   b. Absolute neutrophil count (ANC)<1.0×10⁹/L
   c. Myeloblasts ≥5% in peripheral blood
   d. Serum creatinine clearance <30 mL/min (as per the Modification of Diet in Renal Disease [MDRD] formula)
   e. Serum amylase and lipase >1.5×ULN
   f. Aspartate aminotransferase (AST) or alanine aminotransferase (ALT)>3×upper limit of normal (ULN)
   g. Total bilirubin >1.5×ULN, subject's total bilirubin between 1.5-3.0×ULN are eligible if the direct bilirubin fraction is <25% of the total bilirubin
2. Subject is pregnant or lactating female
3. Subject with previous splenectomy
4. Subject with previous or planned hematopoietic cell transplant
5. Subject with prior history of Wernicke encephalopathy (WE)
6. Subject with signs or symptoms of WE (eg, severe ataxia, ocular paralysis or cerebellar signs) without documented exclusion of WE by thiamine level and brain MRI
7. Subject with thiamine deficiency, defined as thiamine levels in whole blood below normal range according to institutional standard and not demonstrated to be corrected prior to randomization
8. Subject with concomitant treatment with or use of pharmaceutical, herbal agents or food known to be strong inducers of Cytochrome P450 3A4 (CYP3A4), sensitive CYP3A4 substrates with narrow therapeutic range, sensitive Cytochrome P450 2C19 (CYP2C19) substrates with narrow therapeutic range, or sensitive Cytochrome P450 2D6 (CYP2D6) substrates with narrow therapeutic range
9. Subject on any chemotherapy, immunomodulatory drug therapy (e.g., thalidomide, interferon-alpha), anagrelide, immunosuppressive therapy, systemic corticosteroids >10 mg/day prednisone or equivalent. Subjects who have had prior exposure to hydroxyurea (e.g., Hydrea) in the past may be enrolled into the study as long as it has not been administered within 14 days prior to randomization
10. Subject has received ruxolitinib within 14 days prior to randomization
11. Subject with previous exposure to Janus kinase (JAK) inhibitor(s) other than ruxolitinib treatment
12. Subject on treatment with aspirin with doses >150 mg daily
13. Subject with major surgery within 28 days prior to randomization
14. Subject with diagnosis of chronic liver disease (e.g., chronic alcoholic liver disease, autoimmune hepatitis, sclerosing cholangitis, primary biliary cirrhosis, hemochromatosis, non-alcoholic steatohepatitis)
15. Subject with prior malignancy other than the disease under study unless the subject has not required treatment for the malignancy for at least 3 years prior to randomization. However, subjects with the following history/concurrent conditions provided successfully treated may enroll: non-invasive skin cancer, in situ cervical cancer, carcinoma in situ of the breast, incidental histologic finding of prostate cancer (T1a or T1b using the tumor, nodes, metastasis [TNM] clinical staging system), or is free of disease and on hormonal treatment only
16. Subject with uncontrolled congestive heart failure (New York Heart Association Classification 3 or 4)
17. Subject with known human immunodeficiency virus (HIV), known active infectious Hepatitis B (HepB), and/or known active infectious Hepatitis C (HepC)
18. Subject with serious active infection
19. Subject with presence of any significant gastric or other disorder that would inhibit absorption of oral medication
20. Subject is unable to swallow capsule
21. Subject has any significant medical condition, laboratory abnormality, or psychiatric illness that would prevent the subject from participating in the study
22. Subject has any condition including the presence of laboratory abnormalities, which places the subject at unacceptable risk if he/she were to participate in the study or any condition that confounds the ability to interpret data from the study
23. Subject has any condition that confounds the ability to interpret data from the study
24. Subject with participation in any study of an investigational agent (drug, biologic, device) within 30 days prior to randomization

Study Design. The study includes:
A 28-day Screening Period
2:1 Randomization to fedratinib or best available therapy (BAT)
Stratification at Randomization according to:
   Risk category (DIPSS) Int-1 and Int-2 versus High Risk
   Spleen size by palpation: <15 cm below left costal margin (LCM) versus ≥15 cm below LCM
   Platelets ≥100,000/μL versus platelets <100,000/μL
Study Treatment Period (time on study drug plus 30 days after last dose)
Subjects are allowed to crossover from BAT to the fedratinib arm after the Cycle 6 response assessment or before the Cycle 6 response assessment in the event of a confirmed progression of splenomegaly by MRI/CT scan
A Survival Follow-up Period for progression and survival

The expected duration of study is approximately 5 years, which includes approximately 24-months to fully enroll, and 30 months for treatment and follow-up. The actual duration of the trial will be dependent upon the median treatment duration for subjects.

The End of Trial is defined as either the date of the last visit of the last subject to complete the Survival Follow-up, or the date of receipt of the last data point from the last subject that is required for primary, secondary and/or exploratory analysis, as prespecified in the protocol, whichever is the later date. End of trial is expected approximately 2 years after the last subject is randomized. The trial completes when all key endpoints and objectives of the study have been analyzed. The subjects who remain on active treatment and are continuing to derive benefit may have available to them either a roll-over protocol, or alternative means for providing study drug to them after study closure.

Screening Period. All enrolled subjects will undergo screening procedures during the screening period which must be completed within 28 days prior to the start of study treatment. This will serve to determine study eligibility based on all inclusion and exclusion criteria defined in the protocol. For subjects that are receiving ruxolitinib during the screening period or that have potentially reversible laboratory abnormalities (or other criteria that excludes patient from enrollment) detected during screening, the screening period may be extended to 35 days (additional 7 days). If needed, randomization will be preceded by a taper-off period for previous treatment according to the prescribing information and a washout period for previous treatment, in line with the inclusion and exclusion criteria, which is to be started at least 14 days before the screening MRI/CT scan for the study.

Randomization. Upon confirmation of eligibility, subjects will be randomized 2:1 to one of the following arms:
Arm 1 (fedratinib) will include up to 128 subjects receiving fedratinib 400 mg
Arm 2 (BAT) will include up to 64 subjects receiving best available therapy

Treatment Period. Cycles are defined for administrative purposes as 4-week (28 day) periods irrespective of the assigned treatment arm. Subjects may continue treatment with study treatment until unacceptable toxicity, lack of therapeutic effect, progression of disease or until consent is withdrawn.

The fedratinib dose is 400 mg/day PO (4×100 mg capsules) to be self-administered orally once daily continuously on an outpatient basis, preferably together with an evening meal, the same time each day. In case a dose is missed, the next dose should be taken the following day at the same time of day as previously taken before the dose was missed. Fedratinib is administered as the dihydrochloride monohydrate form (i.e., Compound II).

The most common adverse events associated with fedratinib are hematological and gastrointestinal. Hematological adverse events associated with JAK inhibitors are dose dependent, mechanism-based and their managed through dose reductions, dose interruptions and transfusions.

If a subject does not tolerate fedratinib therapy after 2 dose level reductions from the starting dose, he/she must be withdrawn from the study treatment. If the toxicity does not resolve in the time period as specified in the Dose Modification Schedule table (Table 1), subjects must be withdrawn from the study treatment. Reescalation of doses is possible in certain cases as defined in the Dose Modification Schedule table (Table 1). The daily dose of fedratinib cannot exceed 400 mg/day.

Subjects treated on the Best Available Therapy regimen (BAT) arm will be treated according to local prescribing information. BAT may include any investigator selected treatment and is not limited to approved JAK inhibitors (used according to the prescribing information), chemotherapy (e.g., hydroxyurea), anagrelide, corticosteroid, hematopoietic growth factor, immunomodulating agent, androgens, interferon, and may also include “no treatment” and symptom directed treatment. BAT may not include investigational agents, fedratinib (if approved during the course of study), and hematopoietic stem cell transplantation.

Subjects may crossover from the BAT arm to the fedratinib arm at any time before the Cycle 6 response assessment in the event of a confirmed progression of splenomegaly (by MRI/CT scan) or after the Cycle 6 response assessment. Confirmed progression of splenomegaly is defined as enlargement of spleen volume by MRI/CT scan (within 28 days before crossover) of ≥25% compared to the subject's baseline as assessed by the central imaging laboratory. Subjects on the BAT arm that discontinue treatment before Cycle 6 response assessment without confirmed progression of splenomegaly are allowed to stay on study and eventually cross-over at Cycle 6 response assessment.

The presence of any of the following will exclude a subject from crossover to fedratinib treatment:
1. Any of the following laboratory abnormalities assessed within 28 days before cross-over:
   Platelets <25,000/μL or Platelets <50,000/μL if associated with major bleeding
   Absolute neutrophil count (ANC)<0.5×109/L
   Myeloblasts ≥5% in peripheral blood
   Serum creatinine clearance <30 mL/min (as per the Modification of Diet in Renal Disease [MDRD] formula)
   Serum amylase or lipase >2.0×ULN
   Aspartate aminotransferase (AST) or alanine aminotransferase (ALT)>3×ULN
   Total bilirubin >1.5×ULN, subject's total bilirubin between 1.5-3.0×ULN are eligible if the direct bilirubin fraction is <25% of the total bilirubin
2. Subject with signs indicating transformation/progression to blast phase of myelofibrosis
3. Subject has received ruxolitinib any other JAK inhibitor or hydroxyurea within 14 days prior to crossover
4. Subject with thiamine deficiency, defined as thiamine levels in whole blood below normal range according to institutional standard and not demonstrated to be corrected prior to crossover
5. Subject with signs or symptoms of WE (e.g., severe ataxia, ocular paralysis or cerebellar signs) without documented exclusion of WE by thiamine level and brain MM
6. Subject with concomitant treatment with or use of pharmaceutical, herbal agents or food known to be strong inducers of Cytochrome P450 3A4 (CYP3A4), sensitive CYP3A4 substrates with narrow therapeutic range, sensitive Cytochrome P450 2C19 (CYP2C19) substrates with narrow therapeutic range, or sensitive Cytochrome P450 2D6 (CYP2D6) substrates with narrow therapeutic range
7. Subject with serious active infection

All subjects will be monitored for adverse events during the study. All subjects discontinued from protocol-prescribed therapy for any reason will be followed for a period of 30 days following the last dose of study drug to collect safety data.

The average treatment period for each subject in the fedratinib arm is expected to be approximately 12 months. Subjects receiving BAT may crossover to fedratinib treatment at any time before the Cycle 6 response assessment in the event of a confirmed progression of splenomegaly (by MRI/CT scan) or after the Cycle 6 response assessment. The actual study duration for an individual subject will be dependent upon the actual treatment duration and Survival Follow-up duration and is expected not to exceed 5 years.

A flexible dose modification regimen may be employed to minimize drug toxicity for individual subjects, with possible daily doses of 200 mg, 300 mg, or 400 mg. For subjects with severe impairment of renal function and co-administration of strong or moderate CYP3A4 inhibitors the fedratinib dose is adjusted, discussed infra.

Dose Modification Schedule for Fedratinib

The most common adverse events associated with fedratinib are hematological and gastrointestinal events. Hematological adverse events associated with JAK inhibitors are dose dependent, mechanism-based and are managed through dose reductions, dose interruptions and transfusions.

If a subject experiences a drug toxicity as specified in Table 1 below, the dosing must be interrupted; in some cases (i.e., when it is not a liver function test (LFT) abnormality) the dose can be titrated by a 100 mg/day decrement during the study, depending upon the Investigator's judgment, down to a minimum dose of 200 mg/day.

If a subject does not tolerate fedratinib therapy after 2 dose level reductions from the starting dose, he/she must be withdrawn from the study treatment. If the toxicity does not resolve in the time period as specified in Table 1 below, subjects must be withdrawn from the study treatment. Reescalation of doses is possible in certain cases. The daily dose of fedratinib cannot exceed 400 mg/day (based on the free base weight).

TABLE 1
Fedratinib Dose Modification Schedule

| | | | |
|---|---|---|---|
| | Fedratinib | | Fedratinib Dose |
| Adverse Event | Management | Recovery | After Recovery |

Hematological

| | | | |
|---|---|---|---|
| Grade 4 or Grade 3 | Hold fedratinib up to | Grade ≤3 | Dose decrement by |
| thrombocytopenia | 28 days | thrombocytopenia | 1 dose level: 100 |
| with major bleeding | | without bleeding | mg/daily decrease |
| Grade 4 neutropenia | Hold fedratinib up to | Grade ≤2 | Dose decrement by 1 dose |
| | 28 days | neutropenia | level: 100 mg/daily |
| | | | decrease |
| Grade 4 | — | Toxicity | Subsequent upward dose |
| hematological | | resolves for | titration possible of 1 dose |
| toxicity with dose | | at least 1 | level (100 mg daily) per |
| reduction in | | cycle | cycle as per the |
| subsequent cycle | | | Investigator's discretion |
| Recurrence of a grade | — | — | Subsequent upward dose |
| 4 hematological | | | titration not permitted |
| toxicity | | | Fedratinib discontinuation |
| | | | as per the Investigator's |
| | | | discretion |

Non-hematological

| | | | |
|---|---|---|---|
| Drug-related non- | — | — | Subsequent upward dose |
| hematological Grade | | | titration not permitted |
| 4 or unmanageable | | | Fedratinib discontinuation |
| Grade 3 toxicity with | | | as per the Investigator's |
| dose reduction in | | | discretion |
| subsequent cycle | | | |

Hepatic (LFT abnormalities)

| | | | |
|---|---|---|---|
| Grade ≥3 AST or | Hold fedratinib | Grade ≤1 | Fedratinib Hold ≤14 days: |
| ALT or total bilirubin | Weekly monitoring of | | Dose decrement by 1 dose |
| | LFTs, until resolution, | | level: 100 mg daily |
| | After fedratinib resumed, | | decrease |
| | LFT monitoring every | | Subsequent upward dose |
| | 2 weeks for the 3 | | titration not permitted |
| | subsequent cycles | | Fedratinib Hold >14 days |
| | at a minimum | | (AE did not return to |
| | | | Grade ≤1): fedratinib |
| | | | permanently discontinued |
| | | | Grade 4 in the absence of |
| | | | demonstrable cause: |
| | | | permanently discontinue |
| | | | fedratinib |
| Recurrence of LFT | Discontinue fedratinib | — | — |
| abnormality (i.e. ≥ | permanently | | |
| Grade 3 toxicity) | | | |
| after dose reduction | | | |

Gastrointestinal

| | | | |
|---|---|---|---|
| Grade 2 nausea, | Hold fedratinib up to | Toxicity | Consider resuming the |
| vomiting, diarrhea, or | 14 days | resolves to | dose at the same |
| constipation that does | | Grade ≤1 | level after |
| not respond to | | | resolution of |
| adequate therapeutic | | | adverse event |
| or supportive | | | |
| measures within 48 hours | | | |
| Grade ≥3 or | Hold fedratinib up to | Toxicity | Consider reducing one |
| recurrence of Grade 2 | 14 days | resolves to | dose level after |
| nausea vomiting, | | Grade ≤1 | resolution |
| diarrhea, or | | | of adverse event |
| constipation that does | | | |
| not respond to | | | |
| adequate therapeutic | | | |
| or supportive | | | |
| measures within 48 hours | | | |

Other Adverse Events Not Described Above

| | | | |
|---|---|---|---|
| Grade ≥3 or | Hold fedratinib up to | Toxicity | Consider reducing one |
| recurrence of Grade 2 | 14 days | resolves to | dose level after |
| that does not respond | | Grade ≤1 | resolution |
| to adequate | | | of adverse event |
| therapeutic or | | | |
| supportive measures | | | |
| within 48 hours | | | |
| Grade ≥3 non- | Hold fedratinib up to | Toxicity | Dose decrement by |
| hematological | 14 days | resolves to | 1 dose level: |
| toxicity, non- | | Grade ≤1 | 100 mg daily |
| gastrointestinal | | | decrease |
| toxicity or Grade ≥2 | | | |
| peripheral | | | |
| neuropathies | | | |

AE = adverse event;
ALT = alanine aminotransferase;
AST = aspartate aminotransferase;
GI = gastrointestinal;
LFT = liver function test.

Dose Adjustment for Co-Administration with Strong and Moderate CYP3A4 Inhibitors

Concomitant administration of fedratinib with strong or moderate CYP3A4 inhibitors can increase fedratinib exposure. Increased fedratinib exposure may increase the risk of exposure-related AEs and need to be considered carefully.

For subjects with a co-administration of a strong CYP3A4 inhibitor, a dose reduction of the starting dose of fedratinib—from 400 mg to 200 mg—is recommended. In cases where a strong CYP3A4 inhibitor is required to be introduced during treatment, consider dose reduction by 2 decrement dose levels (e.g., from 300 mg to 100 mg). Strong CYP3A4 inhibitors include, but are not limited to, boceprevir, cobicistat, conivaptan, danoprevir and ritonavir, elvitegravir and ritonavir, grapefruit juice, indinavir and ritonavir, itraconazole, ketoconazole, lopinavir and ritonavir, paritaprevir and ritonavir and (ombitasvir and/or dasabuvir), posaconazole, ritonavir, saquinavir and ritonavir, telaprevir, tipranavir and ritonavir, troleandomycin, and voriconazole.

For subjects with a co-administration of a moderate CYP3A4 inhibitor, a dose reduction of the starting dose of fedratinib from −400 mg to 300 mg. In cases where a moderate CYP3A4 inhibitor is required to be introduced during treatment, consider dose reduction by 1 decrement dose level (e.g. from 300 mg to 200 mg). Moderate CYP3A4 inhibitors include, but are not limited to, aprepitant, cimetidine, ciprofloxacin, clotrimazole, crizotinib, cyclosporine, dronedarone, erythromycin, fluconazole, fluvoxamine, imatinib, tofisopam, and verapamil.

If the fedratinib dose needs to be reduced below 100 mg daily based on any fedratinib-related AEs due to a potentially increased plasma concentration of fedratinib, consider a lower average daily dose by administering, for example, 100 mg fedratinib every other day that is equivalent to an average daily dose of 50 mg. If AEs are still not resolved after reducing fedratinib dose, consider interrupting dosing of either fedratinib or strong CYP3A4 inhibitors based on overall benefit/risk for a patient. In cases where co-administration with the CYP3A4 inhibitor is discontinued, the fedratinib dose should be re-escalated accordingly.

Dose adjustment for renal impairment. No dose adjustment is recommended in subjects with mild to moderate renal impairment. In subjects that develop severe renal impairment during the study, the fedratinib dose should be adjusted by one dose decrement level (e.g., from 400 mg to 300 mg once a day [QD]). Subjects on a planned dose of 200 mg QD are allowed to reduce to 100 mg.

Peripheral blood and serum will be collected for exploratory evaluation of mutations, cytokines and circulating blood cell profiles at baseline, as well as during treatment to evaluate pharmacodynamic effects of fedratinib. Pharmacodynamic measures may include inflammatory cytokines (eg, tumor necrosis factor-α [TNF-α], interleukin-12 [IL-12]), immunomodulatory cytokines (eg, IL-2, IL-6, IL-8 and IL-15) (Tefferi A, Pardanani A. JAK inhibitors in myeloproliferative neoplasms: rationale, current data and perspective. Blood Rev. 2011 September; 25(5):229-37), fibrosis markers (e.g., transforming growth factor-β [TGF-β]), signaling pathways, gene expression and/or other molecular markers. Mutation profiles in blood at study entry will be evaluated to classify prognostic risk of patients. Mutation profiles during treatment will be evaluated to assess molecular changes associated with response and relapse to fedratinib therapy. Pharmacodynamic effects will also be evaluated in association with response and relapse to fedratinib therapy.

Overview of Key Efficacy Assessments.

Assessment of Spleen Size. Spleen volume will be assessed at the study site (MM or CT Scan if Mill is contraindicated) during screening and at the end of cycle 3, 6, 12, 18, 24 and at End of Treatment Visit. MM/CT scans will be reviewed centrally. The central review will be blinded for arm assignment and treatment.

Spleen size will also be assessed by palpation at screening and on Day 1 of each treatment cycle, at the end of treatment visit and at the 30-day follow-up visit after last dose of fedratinib.

Assessment of MF-associated symptoms. The MF-related symptoms evaluation will be performed using the MFSAF version 4.0 using a 7-day recall period (Gwaltney C, Paty J, Kwitkowski V E, Mesa R A, Dueck A C, Papadopoulos E J, et al. Development of a harmonized patient-reported outcome questionnaire to assess myelofibrosis symptoms in clinical trials. Leuk Res. 2017 August; 59:26-31).

Overview of Key Safety Assessments. Safety of fedratinib is evaluated based on the incidence of treatment-emergent adverse events (TEAEs) and changes in clinical laboratory parameters, Eastern Cooperative Oncology Group (ECOG) Performance Score (PS), electrocardiogram (ECG), and vital signs.

Safety assessments will comprise:
Record of Adverse Events (AEs) and Serious Adverse Events (SAEs) at each study visit
Physical examination including assessment of abnormal eye movements, cerebellar abnormalities, body weight
Vital signs
Cognitive assessment: Mini-Mental State Examination (MMSE)
Laboratory assessment: hematology, serum chemistry, thiamine level, coagulation, urinalysis, serum/urine pregnancy tests
Electrocardiogram (ECG)

Overview of Other Patient Reported Outcomes. The HRQoL/PRO evaluations will be performed using the following tools:
HRQoL and its various domains will be assessed using the European Organization for Research and Treatment of Cancer Quality of Life C30 (EORTC QLQ-C30) questionnaire version 3 (Aaronson et al, 1993).
Health utilities will be assessed using EQ-5D-5L classifier for 5 levels. The instrument contains 5 items that assess mobility, self-care, usual activities, pain/discomfort, and anxiety/depression and a visual analog scale (VAS) for global health.
The 5 selected treatment-related symptoms from the subject perspective (diarrhea, nausea, vomiting, dizziness, headache) will be assessed by the PRO-CTCAE.

All HRQoL/PRO evaluations will be performed at the site on Day 1 of each treatment cycle, at the End of Treatment (EOT) and the 30-day follow-up visit after last dose of study treatment. All these QoL-related assessments should be performed before any other assessments are performed by the Investigator or designee during the visit.

TABLE 2
Study Endpoints

| | | | Assessment |
|---|---|---|---|
| Endpoint | Name | Description | Timeframe |
| Primary | Spleen volume | Proportion of subjects who have | From Screening to |
| | response rate (RR) | a ≥35% reduction in spleen | the end of Cycle 6 |
| | | volume (SVR) at end of cycle 6 | |
| Key | Symptom response | Proportion of subjects with ≥50% | From C1D1 to the |
| Secondary | rate (SRR) | reduction in total symptom | end of Cycle 6 |
| | | scores measured by MFSAF at | |
| | | end of cycle 6 | |
| | Spleen volume | Proportion of subjects who | From Screening to |
| | response rate | have ≥25% reduction in spleen | the end of Cycle 6 |
| | (RR25) | volume at the end of cycle 6 | |
| Secondary | Safety profile of | Incidence and severity of all | From ICF signature |
| | fedratinib | Grade AEs per NCI CTCAE | up until 30 days after |
| | | Incidence and severity of Grade | last dose |
| | | 3-4 AEs as per the NCI CTC, | For fedratinib related |
| | | including laboratory parameters | AEs, anytime until |
| | | | the last study visit |
| | Spleen response | Proportion of subjects who | From C1D1 to the |
| | rate by palpation | have ≥50% reduction in spleen | end of Cycle 6 |
| | (RRP) | size by palpation at end of | |
| | | cycle 6 | |
| | Durability of spleen | Duration of ≥35 % reduction in | From screening to |
| | response (DR) by | spleen volume | the End of Treatment |
| | MRI | | visit |
| | Durability of spleen | Duration of ≥50% reduction in | From C1D1 until the |
| | response by | spleen size by palpation for | 30-day follow-up |
| | palpation (DRP) | subjects with a palpable spleen at | after last dose visit |
| | | least 5 cm below the left costal | |
| | | margin (LCM) at baseline | |
| | Durability of | Duration of ≥50% reduction in | From C1D1 until the |
| | symptoms response | total symptom scores measured | 30-day follow-up |
| | (DSR) | by MFSAF | after last dose visit |
| | Spleen and disease | Time from randomization to | From randomization |
| | progression free | death due to any reason or | to the End of |
| | survival (SDPFS) | disease progression | Survival Follow-up |
| | | (modified IWG-MRT 2013 | |
| | | including ≥25% increase in | |
| | | spleen volume by MRI/CT) | |
| | Assessment of the | Incidence of patients with a | From ICF signature |
| | effectiveness of risk | CTCAE grade ≥3 of nausea, | to the 30-day follow- |
| | mitigation strategy | diarrhea, or vomiting, or | up after last dose |
| | for gastrointestinal | occurrence of WE (confirmed by | visit |
| | adverse events and | brain MRI or autopsy). | |
| | potential Wernicke | Assessment of thiamine levels at | |
| | encephalopathy | screening, on Day 1 of the first 3 | |
| | (WE) | cycles and every third cycle | |
| | | thereafter, and at the End of | |
| | | Treatment visit | |
| | HRQoL measured | Mean changes in the HRQoL | From C1D1 to Day 1 |
| | by EORTC QOL- | function and symptom domain | of each cycle, at the |
| | C30 domains | scores over the study compared | End of Treatment |
| | | with baseline | visit up to the 30-day |
| | | | follow-up after last |
| | | | dose visit |
| | PRO measured by | Mean changes in the health utility | From C1D1 to Day 1 |
| | EQ-5D-5L | scores over the study compared | of each cycle, at the |
| | | with enrollment measured by | End of Treatment |
| | | EQ-5D-5L | visit up to the 30-day |
| | | | follow-up after last |
| | | | dose visit |
| | Overall Survival | Time from randomization to | From randomization |
| | | death due to any reason | to the End of |
| | | | Survival Follow-up |
| | | | visit |
| Exploratory | Time to spleen | Time from baseline to a ≥50% | From C1D1 to spleen |
| | response by | reduction in spleen size by | response |
| | palpation (TTR) | palpation for subjects with a | |
| | | palpable spleen at least 5 cm | |
| | | below the LCM at baseline | |
| | Best Spleen volume | Best spleen volume response (ml) | From Screening to |
| | response | by MRI/CT scan | the end of Cycle 6 |
| | Pharmacodynamics | Circulating proteins, including | From screening to |
| | | cytokines in blood | End of Treatment |
| | | Hematopoietic cell | visit |
| | | immunophenotypes in blood, | |
| | | including CD34+ cell | |
| | | enumeration, and molecular | |
| | | profiling (eg, gene expression) | |
| | Prognostic markers | Genetic alterations, including | From screening to |
| | | cytogenetics and gene sequencing | End of Treatment |
| | | in blood | visit |
| | Population PK and | Nonlinear mixed effects models | From C1D1 to Cycle 6 |
| | exposure-response | characterize concentration-time | |
| | | data and exposure-response | |
| | | relationship | |
| | Treatment-related | Assessment of the 5-selected | From C1D1 to Day 1 |
| | symptoms from the | treatment-related symptoms from | of each cycle, at the |
| | subject's | the subject perspective (diarrhea, | End of Treatment |
| | perspective | nausea, vomiting, dizziness and | visit up to the 30-day |
| | | headache) using the PRO- | follow-up after last |
| | | CTCAE | dose visit |

AEs = adverse events;
C1D1 = cycle 1 day 1;
CT = computed tomography;
CTCAE = Common Terminology Criteria for Adverse Events;
EORTC QLQ-C30 = European Organization for Research and Treatment of Cancer Quality of Life instrument;
HRQoL = Health-related Quality of life;
ICF = informed consent form;
LCM = left costal margin;
MFSAF = Myelofibrosis Symptom Assessment Form;
MRI = magnetic resonance imaging;
NCI = National Cancer Institute;
PK = pharmacokinetics;
PRO = patient reported outcome;
PRO-CTCAE = Patient Reported Outcome Version of Common Terminology Criteria of Adverse Events;
WE = Wernicke encephalopathy

Efficacy Analysis.

ITT population: this population will consist of all subjects who were randomized. This is the primary analysis population for efficacy variables. All analyses using this population will be based on the treatment assigned by Interactive Response Technology (IRT).

Spleen volume response rate (35%) by MRI/CT. The primary analysis for spleen volume response rate by MRI/CT will be based on ITT population. The data cut-off for RR will occur when the last randomized subject has completed 6 cycles of fedratinib or BAT. Subjects with a missing MRI/CT spleen volume at the end of cycle 6 including those meet the criteria for progression of splenomegaly before end of cycle 6 will be considered non-responders. For crossover subjects, only data before crossover will be included. A Cochran-Mantel-Haenszel (CMH) test will be performed to compare fedratinib to BAT at a one-sided 2.5% alpha level. The RRs and 95% confidence intervals (CI) will be provided for each arm as well as for the difference in RRs and 95% confidence interval of the difference for fedratinib to BAT. In addition, a descriptive summary of spleen volumes measurements and percentage change from baseline will be provided.

Spleen volume response rate (25%) by MRI/CT. The proportion of subjects who have ≥25% reduction in spleen volume at the end of cycle 6 (RR25) is a key secondary endpoint and will be summarized using the same method as RR. Subjects with a missing MRI/CT spleen volume at the end of cycle 6 including those meet the criteria for progression of splenomegaly before end of cycle 6 will be considered non-responders. For crossover subjects, only data before crossover will be included. The analysis will be conducted using ITT and efficacy evaluable population (the subset of ITT population subjects who have been treated and have evaluable spleen volume measurements based on MM/CT scan at baseline and at least one post baseline response assessment by MRI/CT scan. All analyses using this population will be based on the actual treatment received. This population will be used as a secondary analysis population for the primary and selected secondary efficacy variables).

Spleen response rate by palpation (RRP). Spleen response rate by palpation is the proportion of subjects with a spleen response according to the IWG-MRT 2013 at the end of cycle 6 as compared to baseline. This will be calculated for subjects that have an enlarged spleen (≥5 cm below LCM) at baseline. Subjects with a missing spleen size assessment at the end of cycle 6 including those meet the criteria for progression of splenomegaly before end of cycle 6 will be considered not to be responders. The RR by palpation and 95% CI will be provided for each arm as well as for the difference and 95% CI of the difference for fedratinib to BAT. The analysis will be conducted based on ITT populations.

Symptoms response rate (SRR). The SRR is a key secondary endpoint and is defined as the proportion of subjects with ≥50% reduction from baseline to the end of Cycle 6 in total symptom score (TSS) measured by MFSAF version 4.0. Subjects without a baseline TSS>0 will be considered non-evaluable (due to no place for symptom reduction) for the SRR analysis. Subjects with a missing TSS at the end of cycle 6 or who had disease progression before the end of the cycle 6 will be considered non-responder. For crossover subjects crossing over before the End of Cycle 6 assessment, only data before the crossover date will be included for the comparison with fedratinib arm. A CMH test will be performed to compare fedratinib to BAT at a 1-sided 2.5% alpha level. The proportions and 95% CIs will be provided for each arm as well as for the difference in proportions and 95% CI of the difference for fedratinib to BAT. For crossover subjects, the SRR during fedratinib period will be summarized separately using the same method as described above. No formal statistical testing will be conducted to compare with BAT. The analysis will be based on crossover efficacy population with evaluable TSS at time of crossover.

Durability of spleen response by palpation. Duration of spleen response by palpation (DRP) is defined as time from the first documented palpable response according to the IWG-MRT 2013 to the time of the first documented loss of response according to the IWG-MRT 2013. Durability of spleen response by palpation according to the IWG-MRT 2013 criteria will be calculated for subjects that have an enlarged spleen at baseline (≥5 cm below LCM), and that have a spleen response by palpation. In the absence of an event (i.e., no loss of spleen response by palpitation) before the analysis is performed, the DRP will be censored at the date of the last valid assessment performed before the analysis performed date. For crossover subjects without an event, the DR will be censored at the date of the last valid assessment before date. Duration of spleen response by palpation will be analyzed using Kaplan-Meier method. K-M estimates of the 25th, 50th, and 75th percentiles and the 95% confidence interval of median will be provided. K-M curves will be plotted. The analysis will be conducted based on ITT populations.

Durability of spleen volume response by MRI/CT. Duration of spleen volume response (DR) by MRI/CT is defined as time from the first documented spleen response (ie, ≥35% reduction in spleen volume) to the first documented spleen volume reduction <35%. In the absence of an event (i.e. subsequent spleen volume reduction <35%) before the analysis is performed, the DR will be censored at the date of the last valid assessment performed before the analysis performed date. For crossover subjects without an event, the DR will be censored at the date of the last valid assessment before crossover date. Duration of spleen volume response by MRI/CT scan will be analyzed using Kaplan-Meier method. K-M estimates of the 25th, 50th, and 75th percentiles and the 95% confidence interval of median will be provided for both fedratinib and BAT arms. K-M curves will be plotted.

Durability of symptoms response (DSR). The DSR is defined as time from the first documented response in TSS (i.e., reduction in TSS ≥50%) measured by MFSAF version 4.0 to the first documented TSS reduction <50%. In the absence of TSS reduction <50% before the analysis performed, the DSR will be censored at the date of the last valid assessment performed before the analysis performed date. DRS will be analyzed using Kaplan-Meier (K-M) method. K-M estimates of the 25th, 50th, and 75th percentiles and the 95% confidence intervals of median will be provided. K-M curves will be plotted.

Total symptom score (TSS). The TSS is defined as the sum of each of the 7 symptom scores (Gwaltney C, Paty J, Kwitkowski V E, Mesa R A, Dueck A C, Papadopoulos E J, et al. Development of a harmonized patient-reported outcome questionnaire to assess myelofibrosis symptoms in clinical trials. Leuk. Res. 2017 August; 59:26-31). To allow indirect comparison with previous MF studies, a modified TSS (Mesa R A, Gotlib J, Gupta V, Catalano J V, Deininger M W, Shields A L, et al. Effect of ruxolitinib therapy on myelofibrosis-related symptoms and other patient-reported outcomes in COMFORT-I: a randomized, double-blind, placebo-controlled trial. J. Clin. Oncol. 2013 Apr. 1; 31(10):1285-92) will also be derived from the 6 symptoms considered (night sweats, pruritus, abdominal discomfort, early satiety, pain under ribs on left side, bone or muscle pain) and analysis of SRR will be also performed. Fatigue will be assessed as part of the EORTC QLQ-C30. At each timepoint, the TSS (based on 7 symptoms) and the modified TSS will be calculated. Descriptive summary statistics (size, mean, standard deviation, median, range) will be provided for baseline scores, postbaseline scores and change from baseline for TSS, modified TSS and symptom scores.

Spleen and disease progression free survival (SDPFS). Spleen and disease progression free survival is defined as the time from randomization to death due to any reason or disease progression (modified IWG-MRT 2013 including ≥25% increase in spleen volume by MRI/CT). In the absence of an event before the analysis is performed, the SDPFS will be censored at the date of the last valid assessment. For crossover subjects without an event, the SDPFS will be censored at the date of the last valid assessment before crossover date. SDPFS will be analyzed using Kaplan-Meier method. K-M estimates of the 25th, 50th, and 75th percentiles and the 95% confidence interval of median will be provided for both fedratinib and BAT arms. K-M curves will be plotted. The analysis will be conducted based on ITT populations.

Overall Survival. Overall survival (OS) is defined as the time interval from the date of randomization to the date of death due to any cause. In the absence of the confirmation of death before the analysis performed, OS will be censored at the last date of subject was known to be alive or at the study cut-off date (if applicable), whichever is earlier. OS will be analyzed based on the ITT population using Kaplan-Meier (K-M) method. K-M estimates of the 25th, 50th, and 75th percentiles and the 95% confidence intervals of median will be provided for both fedratinib and BAT arms. K-M curves will be plotted.

Crossover Efficacy Analysis. For crossover subjects, the visit cycles will be recounted from cycle 1 for fedratinib exposure period. The analyses during the fedratinib period will be summarized separately using the same method as described above. No formal statistical testing will be conducted to compare with BAT. The analysis will be based on crossover efficacy population, defined as all subjects from BAT arm who crossover to the fedratinib arm.

Exploratory Analysis.

Time to Spleen Response by palpation: time to spleen response by palpation (TTR) is defined as time from randomization to the first documented palpable response (i.e., ≥50% reduction in spleen size by palpation with a palpable spleen at baseline). Time to spleen response by palpation according to the IWG-MRT 2013 criteria will be calculated for subjects that have an enlarged spleen at baseline. In the absence of palpable response before the analysis performed, TTR will be censored at the date of the last valid assessment performed before the analysis performed date. TTR will be analyzed using Kaplan-Meier method. K-M estimates of the 25th, 50th, and 75th percentiles and the 95% confidence interval of median will be provided for both arms. K-M curves will be plotted.

Best Spleen Response Rate by MRI/CT: the best spleen response rate (BRR) during first 6 cycles is defined as proportion of subjects whose spleen volume reduction from baseline ≥35% at any time during first 6 cycles. The BRR and 95% CI will be provided for each arm as well as for the difference in BRR and 95% confidence interval of the difference for fedratinib to BAT.

Survival Follow-up Period. All subjects discontinued from protocol-prescribed therapy for any reason will be followed for survival, subsequent therapies, new malignancy and progression of myelofibrosis to acute myeloid leukemia (AML) every 3 months until death, lost to follow-up, withdrawal of consent for further data collection, or study closure, whichever comes first.

The post-treatment follow-up period will last up to 12 months, and the total expected study duration, including the survival follow-up period, will be approximately 4 years.

Management of Gastrointestinal Adverse Events.

Management of Potential Wernicke Encephalopathy (WE)

A potential case of WE is a medical emergency. Screening for WE and management of potential cases of WE during treatment with fedratinib will be done according the following steps:

Clinical and Cognitive Assessment. Interval history: including a review of the patient's history for confusion, memory problems, vision problems (e.g., double vision) as well as poor nutrition, signs and symptoms of malabsorption, and alcohol use
Physical examination: including assessment for abnormal eye movements, cerebellar abnormalities and body weight (weight loss compared to previous examination or patient history) during screening and Day 1 of every treatment cycle, at the End of Treatment (EOT), and the 30-Day Follow-up visit
Mini-Mental State Examination (MMSE): to objectively assess for signs/symptoms of encephalopathy during screening, on Day 1 of cycles 2 and 3, every 3rd cycle thereafter, at the End of Treatment Visit, and more frequently as clinically indicated

In case of signs or symptoms that may indicate WE:
Hold fedratinib until WE is ruled out
Obtain sample for thiamine level
Empirically start thiamine supplementation
Report the event as an AESI to the Sponsor
Obtain a neurological consult
Perform a brain MRI
If WE is confirmed discontinue fedratinib permanently

Thiamine Monitoring and Correction. Thiamine levels (for whole blood) will be monitored and thiamine supplementation will be administered to all subjects with thiamine levels below the normal range.
Thiamine levels are assessed at screening and need to be corrected and retested before starting fedratinib treatment
While on treatment with fedratinib, thiamine levels are assessed at start of cycles 1, 2, 3 and every 3^{rd} cycle thereafter, at the End of Treatment Visit and as clinically indicated:
   In case a subject is on thiamine supplementation, thiamine levels should be assessed in a fasting state for thiamine supplementation and thiamine given after the blood draw
   In case a thiamine level result is below normal, the site will contact the subject as soon as possible to start thiamine supplementation
   For thiamine levels below the normal range but ≥30 nM/L without signs or symptoms of WE:
      Supplementation with 100 mg oral thiamine must be started
      In case the results were obtained by a local laboratory, report the event as an Adverse Event of Special Interest (AESI) to the Sponsor
   For thiamine level <30 nM/L with or without signs or symptoms of WE:
      Immediate treatment with thiamine (preferably IV), at therapeutic dosages (e.g., 500 mg IV infused over 30 minutes 3 times daily for 2 to 3 days or alternatively IM in equivalent doses according to local standard of care);
      Followed by administration of 250 mg to 500 mg IV thiamine infused once a day for 3 to 5 days or alternatively IM in equivalent doses according to local standard of care; and
      Continue to administer an oral daily dose of 100 mg thiamine for at least 90 days
      Report the event as an Adverse Event of Special Interest (AESI) to the Sponsor
      fedratinib must be held until thiamine levels are restored to normal range.
   Thiamine supplementation should be administered as a thiamine only formulation.
   If thiamine levels are low, ensure that magnesium levels are normal or corrected if low

An adverse event of special interest (AESI) is one of scientific and medical interest specific to understanding of the Investigational Product and may require close monitoring and rapid communication by the Investigator to the sponsor. AESI are to be reported within 24 hours of the Investigator's knowledge of the event via EDC or other appropriate method as directed, if the EDC system is not available, and must be considered an “Important Medical Event” even if no other serious criteria apply; these events must also be documented in the appropriate page(s) of the SAE eCRF in EDC. The rapid reporting of AESIs allows ongoing surveillance of these events to characterize and understand them in association with the use of this investigational product. Events of special interest may be referred to external experts for review as needed.

The following are considered to be Adverse Events of Special Interest (AESI):
Wernicke encephalopathy (WE) or suspected cases of WE associated with thiamine levels below normal range.
Thiamine levels below normal range with or without signs or symptoms of WE
New malignancy after start of study treatment
Progression of myelofibrosis to acute myeloid leukemia (AML)
Grade 3 and 4 hyperlipasemia according to CTCAE criteria, v 5.0
Grade 3 and 4 hyperamylasemia according to CTCAE criteria, v 5.0 or events of pancreatitis
Grade 3 or 4 alanine transaminase (ALT), aspartate transaminase (AST), or total bilirubin elevation or events of hepatotoxicity

Management of Nausea and Vomiting. Management of nausea and vomiting during treatment with fedratinib will be done according the following steps:
Subjects will be provided management instructions (including when to contact the study site) before the start of treatment
In order to mitigate for nausea and vomiting events, it is recommended to take fedratinib with food during an evening meal. Specific instructions for fedratinib administration will be provided for PK sampling days (C1D1, the day before C2D1 and C2D1)
It is highly recommended to use anti-nausea/vomiting treatment prophylactically according to local practice for the first 8 weeks of treatment (e.g., ondansetron). If dimenhydrinate or other muscarinic receptor antagonists are used for nausea and vomiting, administer these agents in the evening to minimize drowsiness and other potential neurological AEs
Hold/reduce the dose of fedratinib according to Table 1
Hospitalization may be indicated for Grade 3 or higher nausea or vomiting or events that persist
For medications that are administered for prophylactic use of nausea and vomiting, if no clinically significant nausea and vomiting occurs during the first 8 weeks of fedratinib treatment, consider weaning the subject off these medications

Management of Diarrhea. Management of diarrhea during treatment with fedratinib will be done according the following steps:
Subjects should have loperamide available at home and should be provided with diarrhea management instructions (including when to contact the study site) before the start of treatment
Loperamide should not be given as prevention in case the subject does not experience diarrhea
Treat with loperamide as per local practice at the onset of diarrhea. Consider starting loperamide at a 4 mg loading dose and then 2 mg after each diarrheal bowel movement without exceeding 16 mg/24 hours
Dietary modifications including adequate hydration, avoidance of lactose containing foods and alcohol, small meals with rice, bananas, bread, etc.
Hold/reduce the dose of fedratinib according to Table 1
Hospitalization may be indicated for Grade 3 or higher persisting diarrhea.
Management of nausea, vomiting and diarrhea will be assessed during the subject's visit on Day 1 of every following 28-day cycle, at Day 15 of the first three cycles and by a mandatory telephone contact at Day 8 of the first cycle.

1. A method of treating a myeloproliferative disorder, comprising administering to a patient previously treated with ruxolitinib Compound I 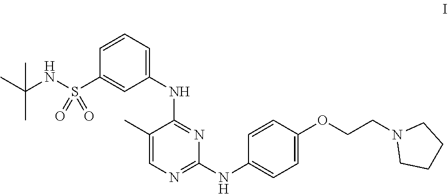 [SMILES: Cc1cnc(Nc2ccc(OCCN3CCCC3)cc2)nc1Nc1cccc(S(=O)(=O)NC(C)(C)C)c1]
or a pharmaceutically acceptable salt or hydrate thereof.

2. The method according to claim 1, wherein the myeloproliferative disorder is resistant or refractory to ruxolitinib.

3. The method according to claim 1 or claim 2, wherein the patient is intolerant to ruxolitinib.

4. The method according to claim 3, wherein the intolerance to ruxolitinib is evidenced by a hematological toxicity or a non-hematological toxicity.

5. The method according to claim 1 or claim 2, wherein the patient has relapsed.

6. The method according to claim 1 or claim 2, wherein the patient has exhibited or experienced one or more of the following during treatment with ruxolitinib: lack of response, disease progression, or loss of response.

7. The method according to claim 6, wherein disease progression is evidenced by an increase in spleen size.

8. A method of reducing spleen volume by at least 25% in a patient suffering from or diagnosed with a myeloproliferative disorder comprising administering to a patient previously treated with ruxolitinib Compound I 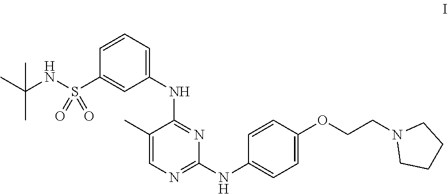 [SMILES: Cc1cnc(Nc2ccc(OCCN3CCCC3)cc2)nc1Nc1cccc(S(=O)(=O)NC(C)(C)C)c1]
or a pharmaceutically acceptable salt or hydrate thereof.

9. The method according to claim 8, wherein the patient's spleen volume is reduced by at least 35%.

10. A method of improving overall survival in a patient suffering from or diagnosed with a myeloproliferative disorder comprising administering to a patient previously treated with ruxolitinib Compound I 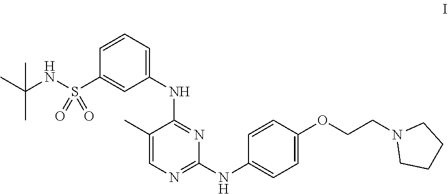 [SMILES: Cc1cnc(Nc2ccc(OCCN3CCCC3)cc2)nc1Nc1cccc(S(=O)(=O)NC(C)(C)C)c1]
or a pharmaceutically acceptable salt or hydrate thereof.

11. A method of improving symptom response rate in a patient suffering from or diagnosed with a myeloproliferative disorder comprising administering to a patient previously treated with ruxolitinib Compound I 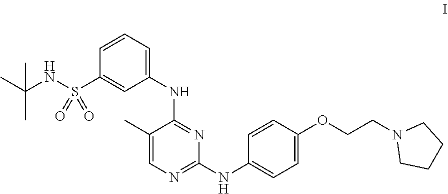 [SMILES: Cc1cnc(Nc2ccc(OCCN3CCCC3)cc2)nc1Nc1cccc(S(=O)(=O)NC(C)(C)C)c1]
or a pharmaceutically acceptable salt or hydrate thereof.

12. The method according to claim 11, wherein symptom response rate is evidenced by at least 50% reduction in total symptom score (TSS).

13. A method of increasing the median survival in a patient population suffering from or diagnosed with a myeloproliferative disorder that has relapsed and/or is refractory to ruxolitinib comprising administering to a patient previously treated with ruxolitinib Compound I 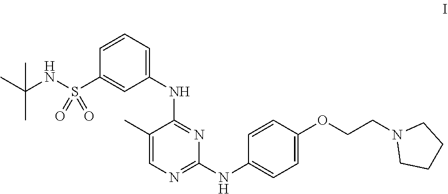 [SMILES: Cc1cnc(Nc2ccc(OCCN3CCCC3)cc2)nc1Nc1cccc(S(=O)(=O)NC(C)(C)C)c1]
or a pharmaceutically acceptable salt or hydrate thereof.

14. A method of decreasing allele burden in a patient having a somatic mutation or clonal marker associated with or indicative of a myeloproliferative disorder comprising administering to a patient previously treated with ruxolitinib Compound I 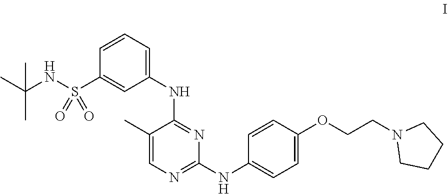 [SMILES: Cc1cnc(Nc2ccc(OCCN3CCCC3)cc2)nc1Nc1cccc(S(=O)(=O)NC(C)(C)C)c1]
or a pharmaceutically acceptable salt or hydrate thereof.

15. The method according to claim 14, wherein the somatic mutation is selected from a JAK2 mutation, a CALR mutation or a MPL mutation.

16. The method according to claim 15, wherein the JAK2 mutation is V617F.

17. The method according to claim 15, wherein the CALR mutation is a mutation in exon 9.

18. The method according to claim 15, wherein the MPL mutation is selected from W515K and W515L.

19. The method according to any of claims 1-18, wherein the myeloproliferative disorder is selected from intermediate risk MPN-associated myelofibrosis and high risk MPN-associated myelofibrosis.

20. The method according to claim 19, wherein the intermediate risk MPN-associated myelofibrosis is selected from primary myelofibrosis, post-polycythemia vera (post-PV) myelofibrosis and post-essential thrombocythemia (post-ET) myelofibrosis.

21. The method according to claim 19, wherein the high risk MPN-associated myelofibrosis is selected from primary myelofibrosis, post-polycythemia vera (post-PV) myelofibrosis and post-essential thrombocythemia (post-ET) myelofibrosis.

22. The method according to any of claims 1-21, wherein Compound I is in the form of a dihydrochloride monohydrate.

23. The method according to any of claims 1-22, wherein the patient has been previously treated with ruxolitinib for at least 3 months.

24. The method according to any of claims 1-22, wherein the patient has been previously treated with ruxolitinib for at least 28 days complicated by
i. development of a red blood cell transfusion requirement; or
ii. Grade ≥3 adverse event(s) of thrombocytopenia, anemia, hematoma, and/or hemorrhage while on treatment with ruxolitinib.

25. The method according to any of claims 1-24, wherein the dose of Compound I, or a pharmaceutically acceptable salt thereof, is about 400 mg, based on the free base weight of Compound I.
